(19) United States
(12) Patent Application Publication
(10) Pub. No.: US 2009/0179196 A1
(43) Pub. Date: Jul. 16, 2009

(54) Pyrene-Based Organic Compound, Transistor Material and Light-Emitting Transistor Device

(76) Inventors: Chihaya Adachi, Fukuoka (JP); Takahito Oyamada, Saitama (JP); Hiroyuki Uchiuzou, Fukuoka (JP); Seiji Akiyama, Kanagawa (JP); Takayoshi Takahashi, Kyoto (JP); Kumiko Takenouchi, Kanagawa (JP); Masaki Shimizu, Kyoto (JP); Tamejiro Hiyama, Kyoto (JP); Etsuya Okamoto, Kyoto (JP)

Correspondence Address: WENDEROTH, LIND & PONACK, L.L.P., 1030 15th Street, N.W.,, Suite 400 East, Washington, DC, 20005-1503, US

(21) Appl. No.: 12/225,370

(22) PCT Filed: Mar. 20, 2007
(86) PCT No.: PCT/JP2007/055603

§ 371 Date: Dec. 19, 2008

(30) Foreign Application Priority Data
Mar. 20, 2006 (JP) 2006-077428
Sep. 20, 2006 (JP) 2006-254360
Feb. 9, 2007 (JP) 2007-030093

(51) Int. Cl. H01L 51/54; C07D 409/14; C07D 209/86; C07D 409/04; C07C 15/02; C07C 15/12; C07C 15/24; C07C 211/54; C07D 401/04; C07D 213/06; C07D 333/08; C07D 307/36

(52) U.S. Cl. 257/40; 549/59; 548/446; 585/24; 585/25; 585/26; 564/433; 546/255; 546/348; 549/80; 549/506; 257/E51.026

(57) ABSTRACT

The object is to provide a light-emitting transistor material which is high in both luminescent property and mobility when used as a light-emitting transistor device.

A light-emitting transistor device is provided in which light emission of a specific pyrene-based organic compound is used for a light-emitting layer of a transistor device.

### TECHNICAL FIELD

The present invention relates to a transistor material which can be used for light-emitting transistor devices, and light-emitting transistor devices using such a material.

### BACKGROUND ART

A light-emitting transistor device is a composite device comprising an organic transistor having a light-emitting function. A device comprising a light-emitting transistor device is made up of a smaller number of parts and thus is more compact in size than conventional devices including a transistor portion and a light-emitting portion that are separate from each other. Because a light-emitting transistor device is considered to be higher in light-emitting efficiency, too, it is gathering attention these days.

As materials usable for light-emitting transistors, non-patent document 1 discloses tetracene, and non-patent document discloses oligothiofuran and polyphenylene vinylene.

Non-patent document 1: Appl. Phys. Lett., 2005, 86, 141106.

Non-patent document 2: Science, 2000, 290, 963.

### DISCLOSURE OF THE INVENTION

### Object of the Invention

But because these materials are low in luminescent properties and charge carrier mobility, further improvements are required.

An object of the present invention is to provide a light-emitting transistor material which is high in both luminescent property and mobility when used as a light-emitting transistor device.

### Means to Achieve the Object

As a result of earnest research, the present inventors discovered that by using, as a transistor material, a compound comprising a pyrene ring as a basic structure, and specific substituents located at specific positions, it is possible to obtain a light-emitting transistor device having extremely high luminescent properties and carrier mobility and thus arrived at the present invention.

In particular, the present invention is directed to a transistor material comprising a compound expressed by one of the below formulas (1) and (2), and a light-emitting transistor containing such a transistor material. The present invention is also directed to novel pyrene-based organic compounds expressed by the below formulas (3), (4) and (5), which are useful as transistor materials.

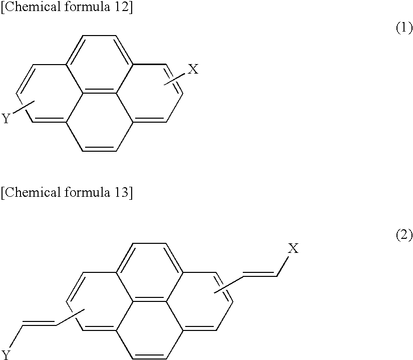

(In the formulas (1) and (2), each of X and Y is a substituent independent of the other and selected from an aromatic hydrocarbon group which may have a substituent, an aromatic heterocyclic group which may have a substituent, an alkyl group which may have a substituent, a cycloalkyl group which may have a substituent, an alkenyl group which may have a substituent, and an alkynyl group which may have a substituent. The substituents which X and Y may have are groups selected from alkyl groups having a carbon number of 1-20, alkoxy groups having a carbon number of 1-20, amino groups, boryl groups, silyl groups, cyano groups, aromatic hydrocarbon groups having a carbon number of 6-36, aromatic heterocyclic groups having a carbon number of 4-36 and halogen atoms. The compound expressed by either of the formulas (1) and (2) includes one pyrene ring in each molecule.)

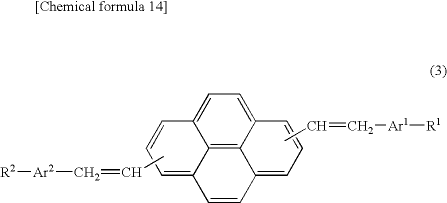

(In the formula (3), Ar¹ and Ar² represent, independently of each other, aromatic hydrocarbon groups other than a pyrene ring or aromatic heterocyclic groups. R¹ and R² represent, independently of each other, halogen atoms, alkyl groups which may have a substituent having a carbon number of 2-20 or alkoxyl groups having a carbon number of 1-20. Each of Ar¹ and Ar² has one or more of R¹ or R². The abovementioned substituent is a group selected from aromatic hydrocarbon groups having a carbon number of 6-36, aromatic heterocyclic groups having a carbon number of 4-36, and halogen atoms.)

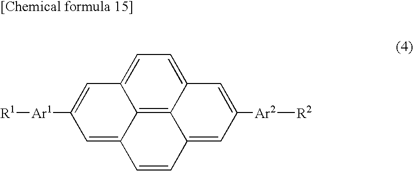

(In the formula (4), Ar¹ and Ar² represent, independently of each other, aromatic hydrocarbon groups other than a pyrene ring or aromatic heterocyclic groups. R¹ and R² represent, independently of each other, halogen atoms, alkyl groups which may have a substituent having a carbon number of 2-20 or alkoxyl groups having a carbon number of 1-20. Each of Ar¹ and Ar² has one or more of R¹ or R². The abovementioned substituent is a group selected from aromatic hydrocarbon groups having a carbon number of 6-36, aromatic heterocyclic groups having a carbon number of 4-36, and halogen atoms.)

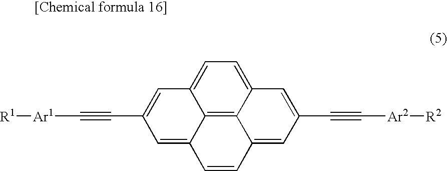

(In the formula (5), Ar¹ and Ar² represent, independently of each other, aromatic hydrocarbon groups other than a pyrene ring or aromatic heterocyclic groups. R¹ and R² represent, independently of each other, halogen atoms, alkyl groups which may have a substituent having a carbon number of 2-20 or alkoxyl groups having a carbon number of 1-20. Each of Ar¹ and Ar² has one or more of R¹ or R². The abovementioned substituent is a group selected from aromatic hydrocarbon groups having a carbon number of 6-36, aromatic heterocyclic groups having a carbon number of 4-36, and halogen atoms.)

### ADVANTAGES OF THE INVENTION

Since the transistor material according to the present invention has high carrier mobility, it is extremely effective as a transistor material. By using the transistor material according to this invention, the crystallinity increases, so that it is possible to improve both the luminescent properties and carrier mobility of the light-emitting transistor device obtained.

### BRIEF DESCRIPTION OF THE DRAWINGS

FIG. 1(a) is a chemical formula showing examples of X and Y of pyrene-based organic compounds.

FIG. 1(b) is a chemical formula showing examples of X and Y of pyrene-based organic compounds.

FIG. 2(a) is a chemical formula showing examples of X and Y of pyrene-based organic compounds.

FIG. 2(b) is a chemical formula showing examples of X and Y of pyrene-based organic compounds.

FIG. 3(a) is a chemical formula showing examples of X and Y of pyrene-based organic compounds.

FIG. 3(b) is a chemical formula showing examples of X and Y of pyrene-based organic compounds.

FIG. 4 is a chemical formula showing examples of X and Y of pyrene-based organic compounds.

FIG. 5 is a chemical formula showing examples of X and Y of pyrene-based organic compounds.

FIG. 6(a) is a chemical formula showing examples of X and Y of pyrene-based organic compounds.

FIG. 6(b) is a chemical formula showing examples of X and Y of pyrene-based organic compounds.

FIG. 7(a) is a chemical formula showing examples of X and Y of pyrene-based organic compounds.

FIG. 7(b) is a chemical formula showing examples of X and Y of pyrene-based organic compounds.

FIG. 8 is a chemical formula showing examples of X and Y of pyrene-based organic compounds.

FIG. 9 is a chemical formula showing examples of X and Y of pyrene-based organic compounds.

FIG. 10 is a chemical formula showing examples of X and Y of pyrene-based organic compounds.

FIG. 11 is a chemical formula showing examples of X and Y of pyrene-based organic compounds.

FIG. 12 is a chemical formula showing examples of X and Y of pyrene-based organic compounds.

FIG. 13 is a chemical formula showing examples of X and Y of pyrene-based organic compounds.

FIG. 14 is a chemical formula showing examples of X and Y of pyrene-based organic compounds.

FIG. 15 is a chemical formula showing examples of X and Y of pyrene-based organic compounds.

FIG. 16 is a chemical formula showing examples of X and Y of pyrene-based organic compounds.

FIG. 17 is a chemical formula showing examples of X and Y of pyrene-based organic compounds.

FIG. 18 is a chemical formula showing examples of X and Y of pyrene-based organic compounds.

FIG. 19 is a chemical formula showing examples of X and Y of pyrene-based organic compounds.

FIG. 20 is a chemical formula showing examples of X and Y of pyrene-based organic compounds.

FIG. 21 is a chemical formula showing examples of X and Y of pyrene-based organic compounds.

FIG. 22(a) is a chemical formula showing examples of X and Y of pyrene-based organic compounds.

FIG. 22(b) is a chemical formula showing examples of X and Y of pyrene-based organic compounds.

FIG. 22(c) is a chemical formula showing examples of X and Y of pyrene-based organic compounds.

FIG. 23 is a sectional view of a light-emitting transistor device according to this invention.

FIG. 24 is a plan view showing the structures of a source electrode and a drain electrode.

FIGS. 25(a) to 25(c) are schematic views showing the light-emitting mechanism of a light-emitting transistor device.

FIG. 26 is a electric circuit diagram of a display device using the light-emitting transistor device according to this invention.

FIG. 27 show chemical formulas of organic fluorescent substances having different wavelengths.

1. Light-emitting layer
2. Source electrode
2a. Comb tooth-shaped region
3. Drain electrode
3a. Comb tooth-shaped region
4. Gate electrode
5. Insulating film
10. Light-emitting transistor device
11. Channel of holes
12. Pinch-off point
20. Substrate
21. Display device
22. Scanning line driving circuit
23. Data line driving circuit
24. Controller
S. Source electrode
D. Drain electrode
G. Gate electrode
C. Capacitor
Ts. Selecting transistor
P11, P12. Pixel
LS1, LS2. Scanning line

### BEST MODE FOR EMBODYING THE INVENTION

The present invention is now described in detail.

The present invention is directed to a transistor material comprising a 2-substituted derivative of which substituted positions are specified. This 2-substituted derivative has high carrier mobility, and can be used as a transistor material used in a device using an organic semiconductor such as an organic field-effect transistor. Because the compound used in the present invention has luminescent properties, it can be used as a light-emitting transistor device.

Specifically, this 2-substituted derivative comprises a compound expressed by the below formula (1) or (2)

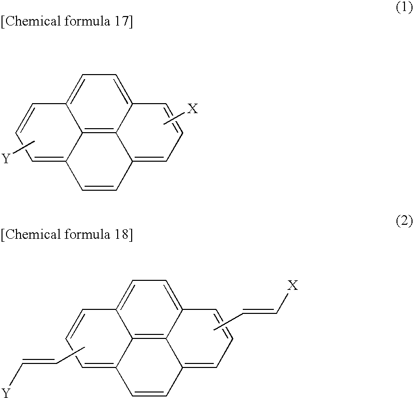

In the formulas (1) and (2), each of X and Y is a substituent independent of the other and selected from an aromatic hydrocarbon group which may have a substituent, an aromatic heterocyclic group which may have a substituent, an alkyl group which may have a substituent, a cycloalkyl group which may have a substituent, an alkenyl group which may have a substituent, and an alkynyl group which may have a substituent. The substituents which X and Y may have are groups selected from alkyl groups having a carbon number of 1-20, alkoxy groups having a carbon number of 1-20, amino groups, boryl groups, silyl groups, cyano groups, aromatic hydrocarbon groups having a carbon number of 6-36, aromatic heterocyclic groups having a carbon number of 4-36 and halogen atoms. The compound expressed by either of the formulas (1) and (2) includes one pyrene ring in each molecule.

Since the transistor material comprising the compound expressed by the formula (1) or (2) contains fewer substituents, and the substituents are located at specific positions, the pyrene ring, which is the basic structure of the compound, is less likely to three-dimensionally interfere with the introduced substituents, and also, the compounds expressed by the formulas (1) and (2) are less likely to three-dimensionally interfere with each other. It is thus presumed that its crystal structure becomes dense, thereby improving the energy transfer efficiency between molecules, which in turn improves the mobility and the light-emitting efficiency of the light-emitting transistor device. Thus, the light-emitting transistor material according to the present invention can be used as a main component of a light-emitting layer of a light-emitting transistor device. These compounds should include only one pyrene ring in each molecule. This is because if they include a pyrene ring as a substituent, the conjugate tends to be too long to achieve visible light emission.

Description is now made of compounds expressed by the formulas (1) and (2).

### (X and Y)

X and Y are substituents independent of each other and selected from an aromatic hydrocarbon group which may have a substituent, an aromatic heterocyclic group which may have a substituent, an alkyl group which may have a substituent, a cycloalkyl group which may have a substituent, an alkenyl group which may have a substituent, and an alkynyl group which may have a substituent.

The aromatic hydrocarbon group is preferably one other than a pyrene ring, such as a phenyl group, biphenyl group, terphenyl group, naphthyl group (preferably 2-naphthyl group), anthryl group (preferably, 2-anthryl group), phenanthryl group, fluorenyl group, or phenyl-etheno-phenyl group. They may have a substituent. Among them, an aromatic hydrocarbon group having a carbon number of not more than 14 is especially preferable. This is because too large a carbon number is not preferable for greater freedom of molecules.

The aromatic heterocyclic group may be a pyridyl group, pyradyl group, bipyridyl group, phenylpyridyl group, pyridino-phenyl group, furyl group, thienyl group, bithienyl group, terthienyl group, pyrolidyl group, imidazole group, benzoimidazole group, oxazole group, indole group, benzoxazole group, thiazole group, benzothiazole group, benzothiazolyl group, benzofuryl group, benzoxazolyl group, pyrrolyl group, pyridazyl group, pyrazinyl group, pyrimidyl group, thienyl group, bithienyl group, phenylthienyl group, benzothienyl group or quinolyl group. They may have a substituent. Among them, an aromatic heterocyclic group having a carbon number of not more than 12 is especially preferable because too large a carbon number is not preferable for greater freedom of molecules.

The alkyl group may be a straight-chain or branched alkyl group having a carbon number of 1 to 20, such as a methyl group, ethyl group, n-propyl group, 2-propyl group, n-butyl group, isobutyl group, tert-butyl group, hexyl group, octyl group, dodecyl group or octadecyl group. They may have a substituent.

The cycloalkyl group may be a cyclohexyl group or a cycloheptyl group. They may have a substituent.

The alkenyl group may be a vinyl group, phenyl-substituted vinyl group, trimethylsilyl-substituted ethynyl groups or propargyl group. They may have a substituent.

The alkynyl group may be an ethynyl group, phenyl-substituted ethynyl group, ethyl-substituted vinyl group, allyl group, or 1-butenyl group. They may have a substituent.

The substituents which X and Y may have may be groups selected from alkyl groups having a carbon number of 1-20, alkoxy groups having a carbon number of 1-20, amino groups, boryl groups, silyl groups, cyano groups, aromatic hydrocarbon groups having a carbon number of 6-36, preferably 6-14, aromatic heterocyclic groups having a carbon number of 4-36, preferably 4-12, and halogen atoms. The same groups as mentioned above for X and Y can be listed as specific ones of these substituents.

X and Y may be different from each other or may be identical to each other, but preferably, X and Y are identical to each other for controllability of the molecular sequence and improved mobility.

X is preferably bonded to the pyrene ring at one of positions 1 to 3, and Y is preferably bonded to the pyrene ring at one of positions 6 to 8. Specifically, the compounds shown by the below formulas (1-1), (1-2), (1-3), (2-1), (2-2) and (2-3) are preferable. Because these compounds are high in symmetric properties, when used as transistor materials, high mobility can be expected. X and Y in the compounds shown by the formulas (1-1), (1-2), (1-3), (2-1), (2-2) and (2-3) are those as described above. Any of the compounds shown by the formulas (1-1), (1-2), (1-3), (2-1), (2-2) and (2-3) have only one pyrene ring in each molecule.

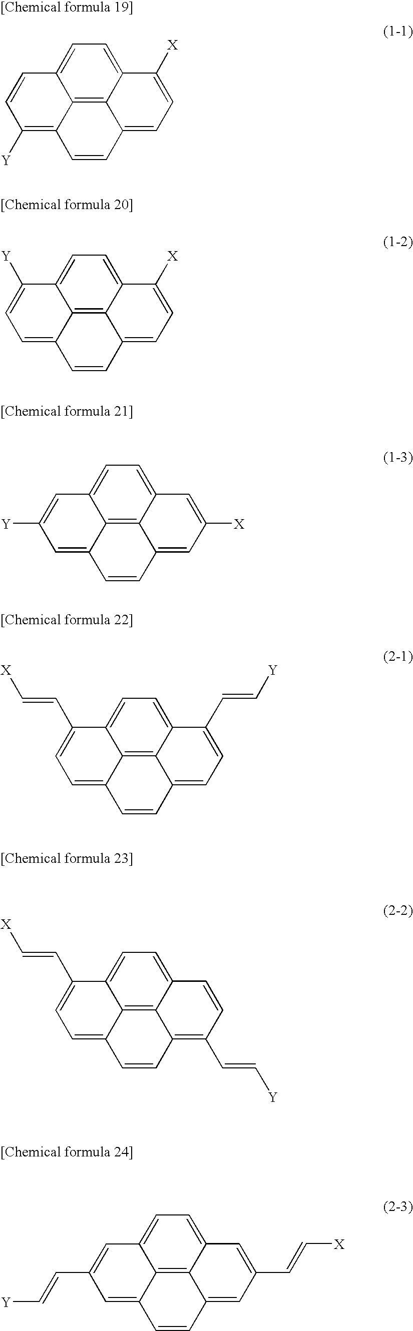

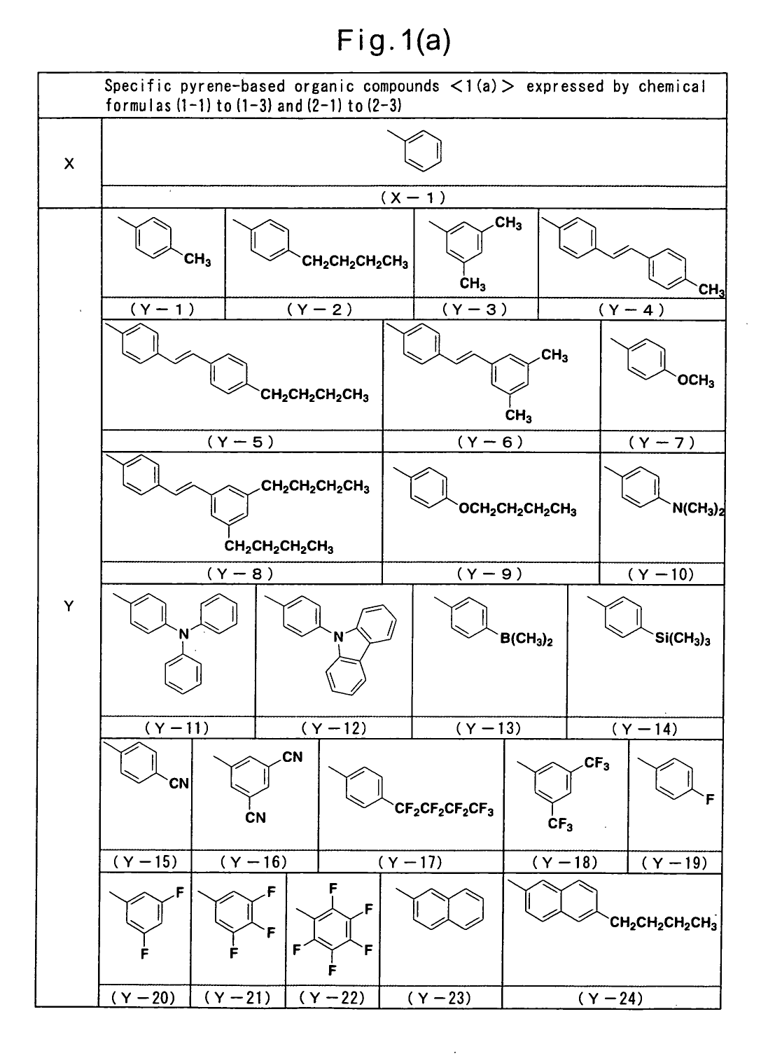

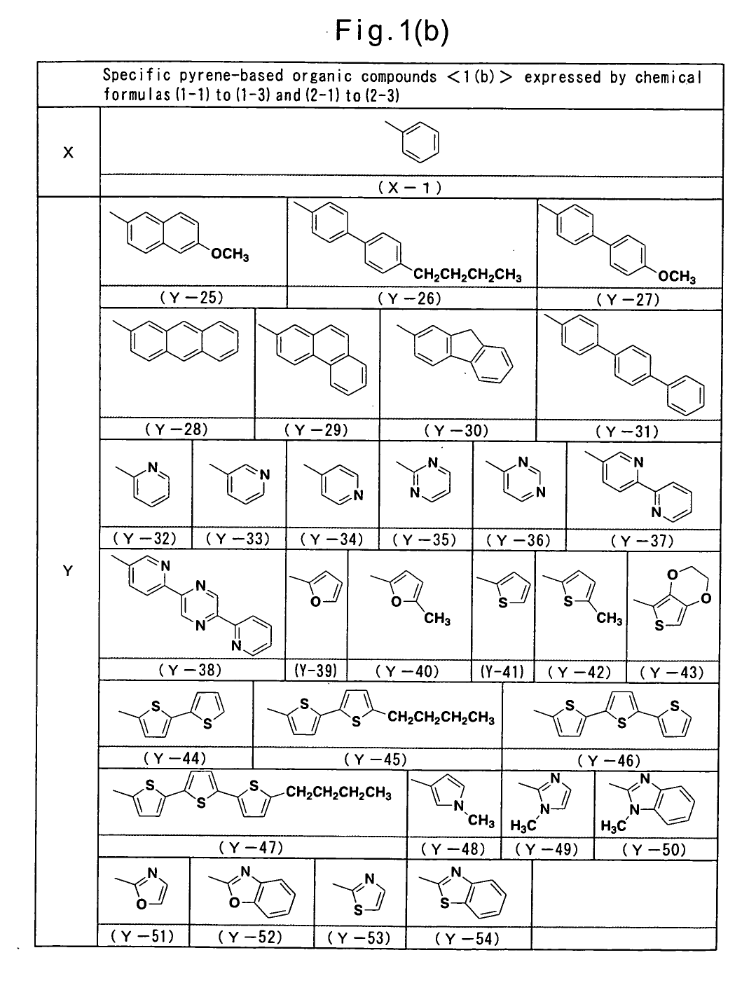

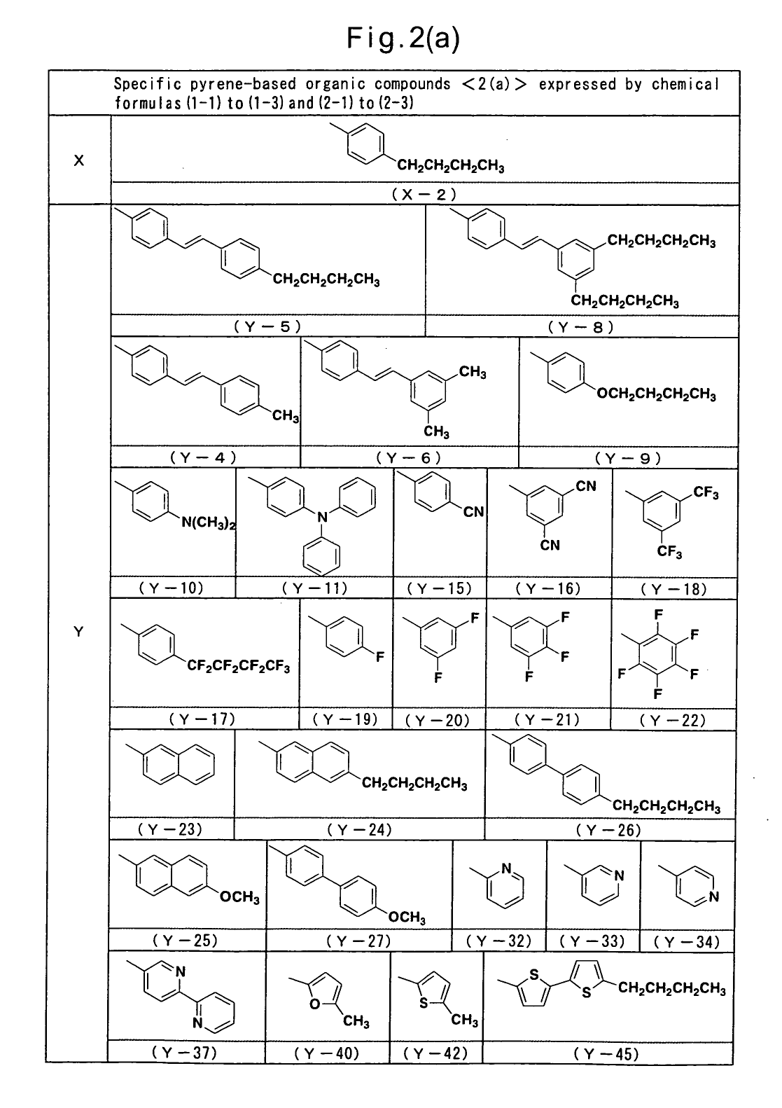

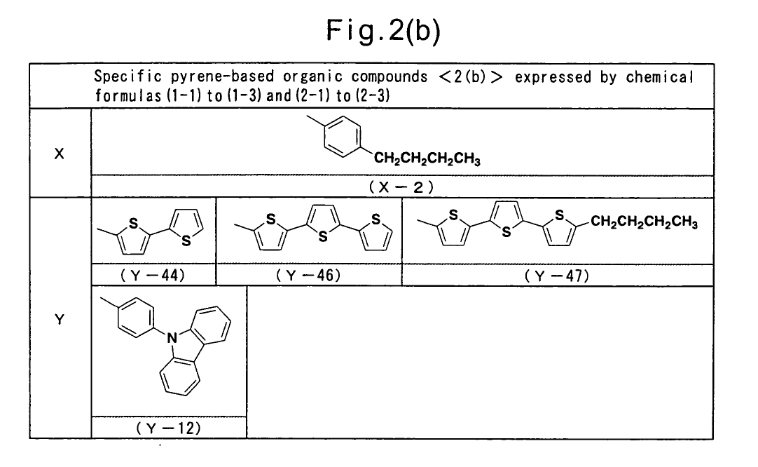

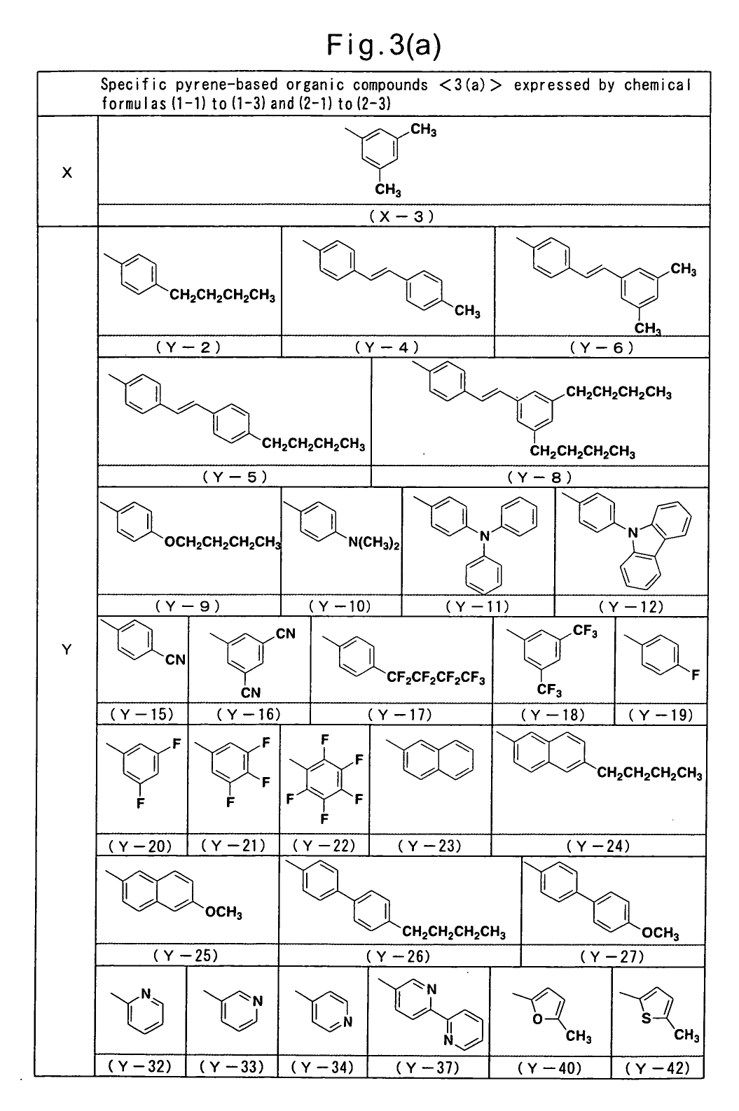

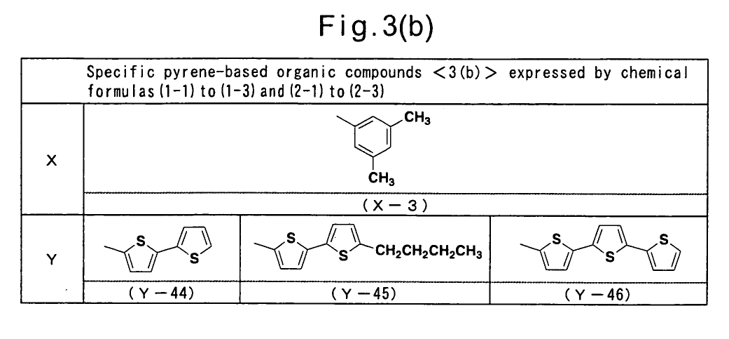

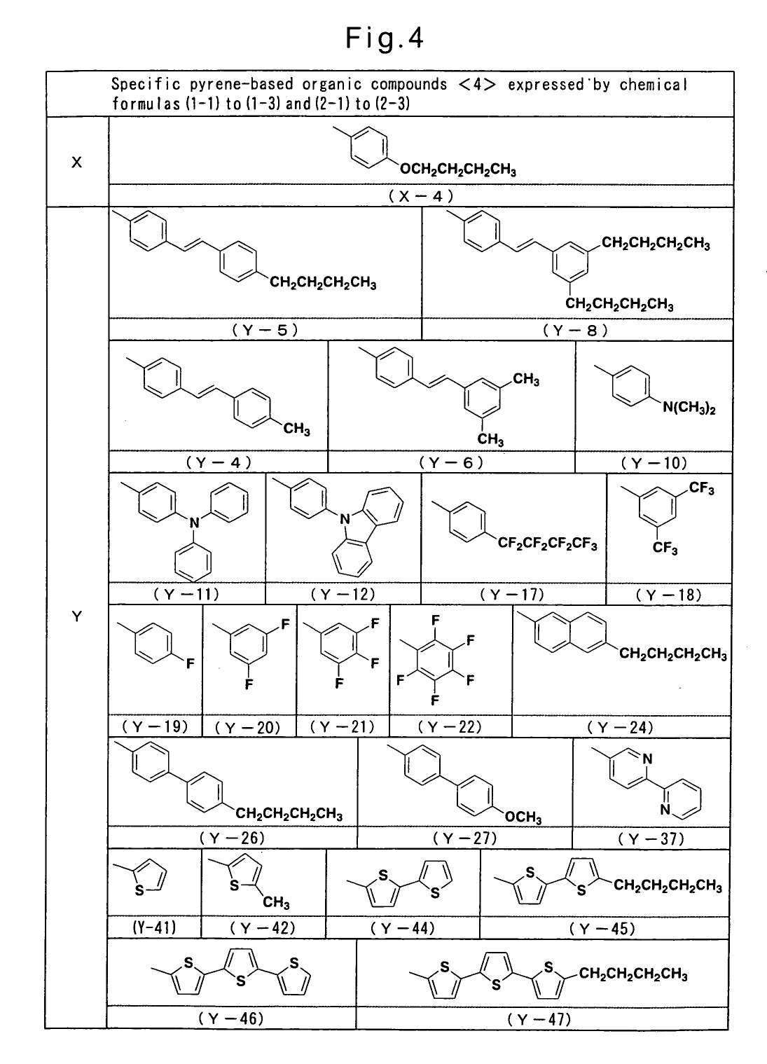

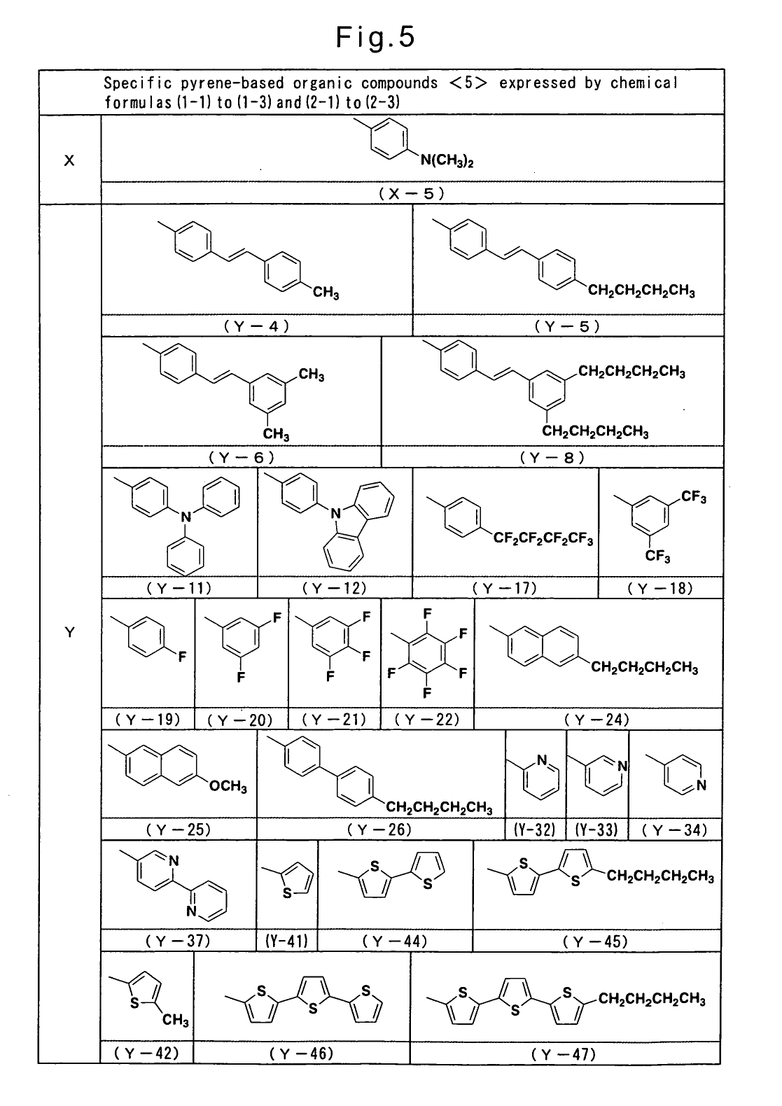

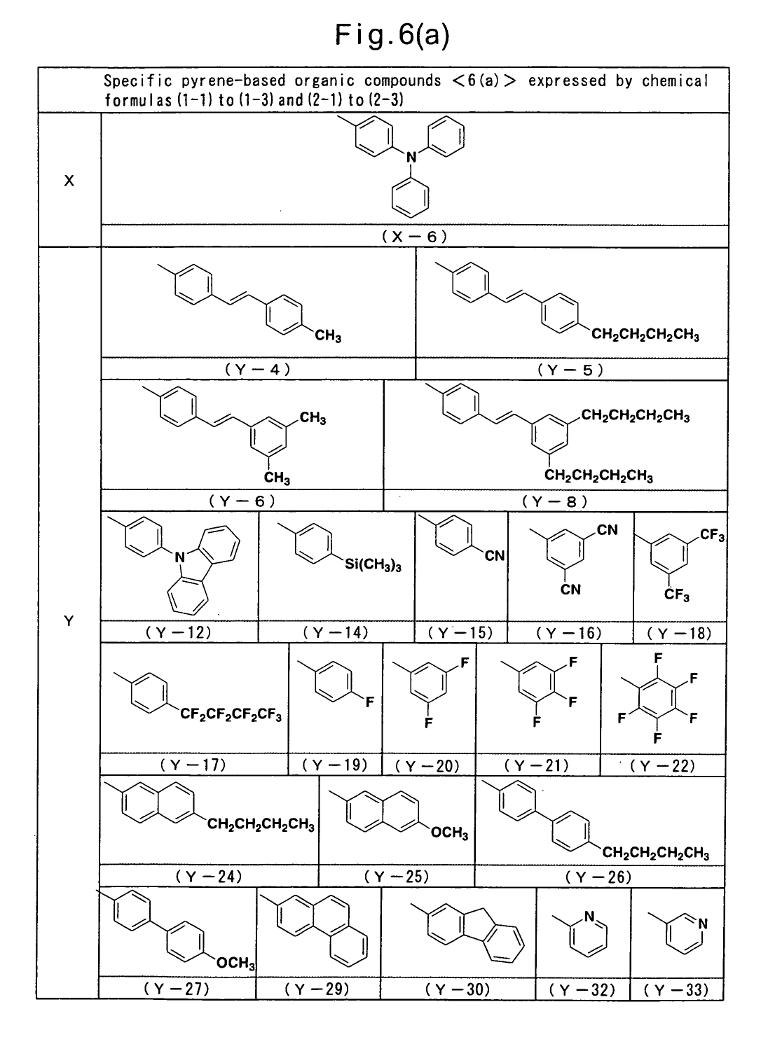

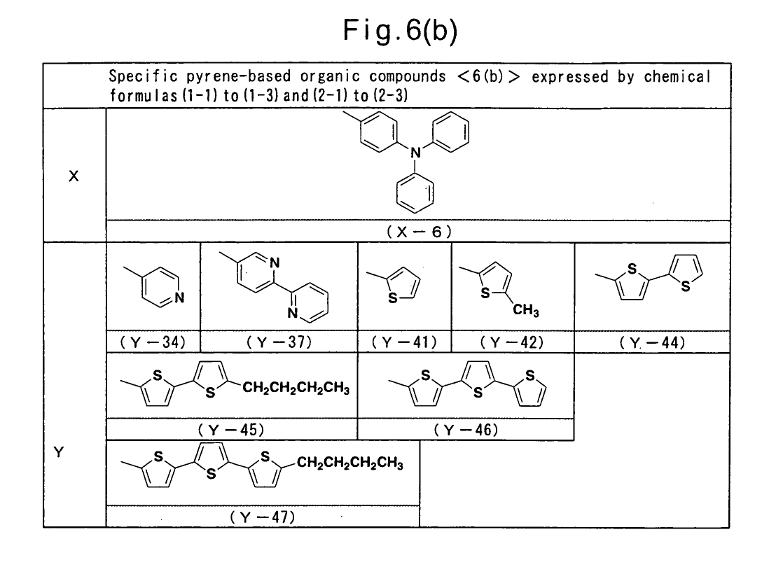

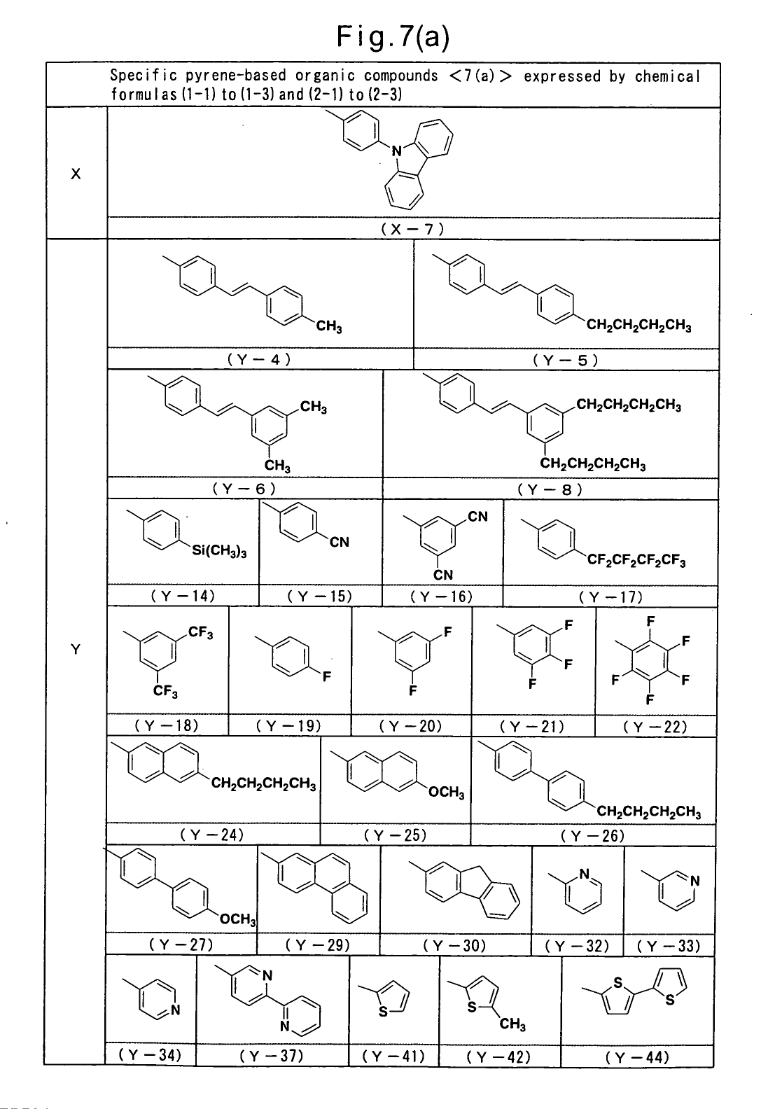

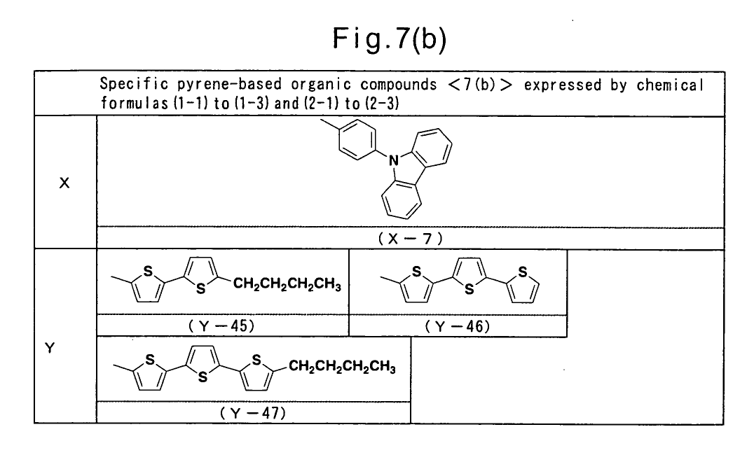

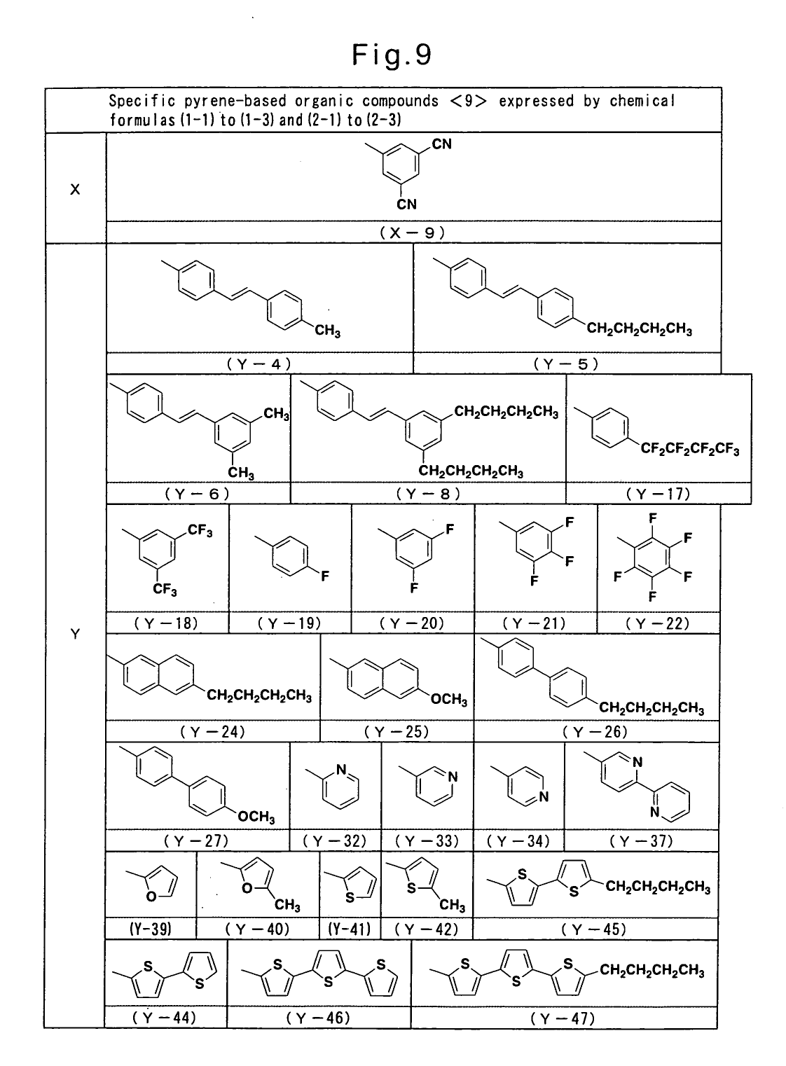

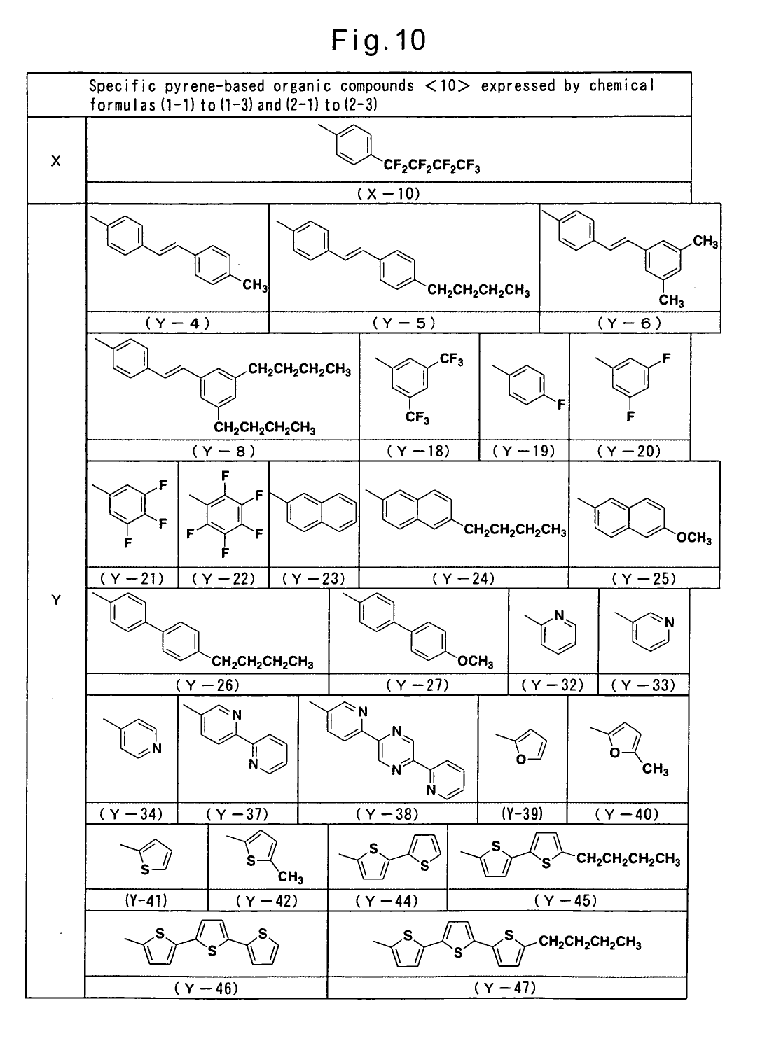

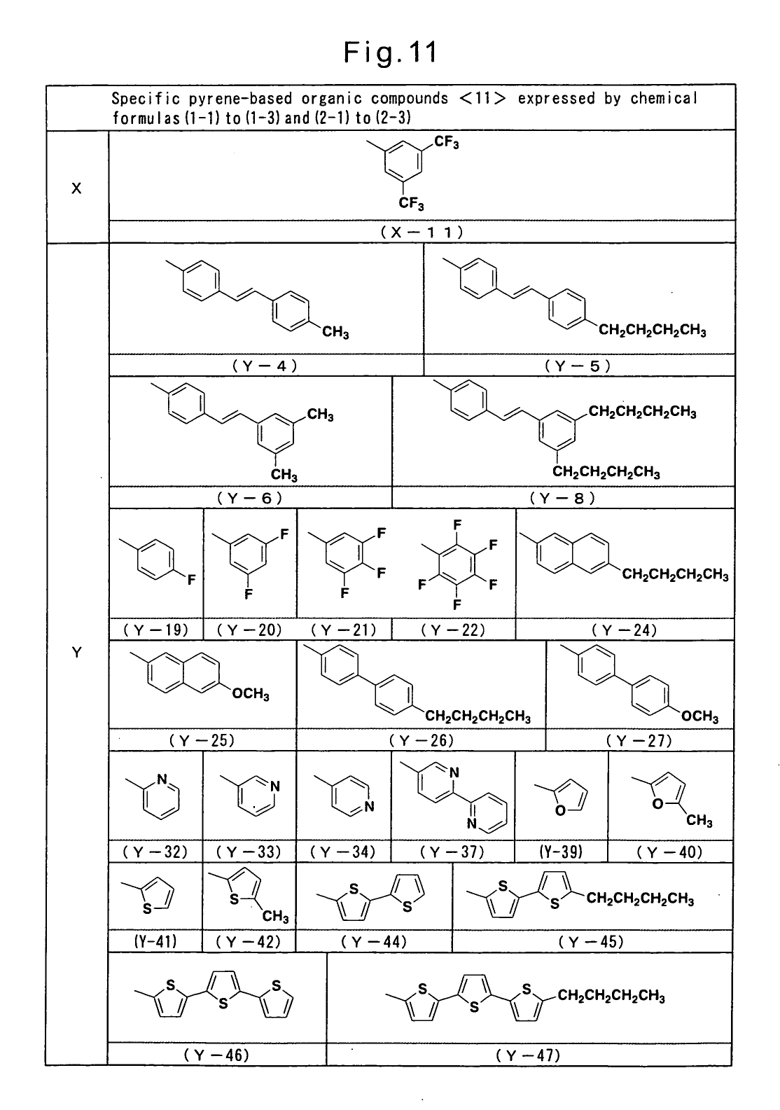

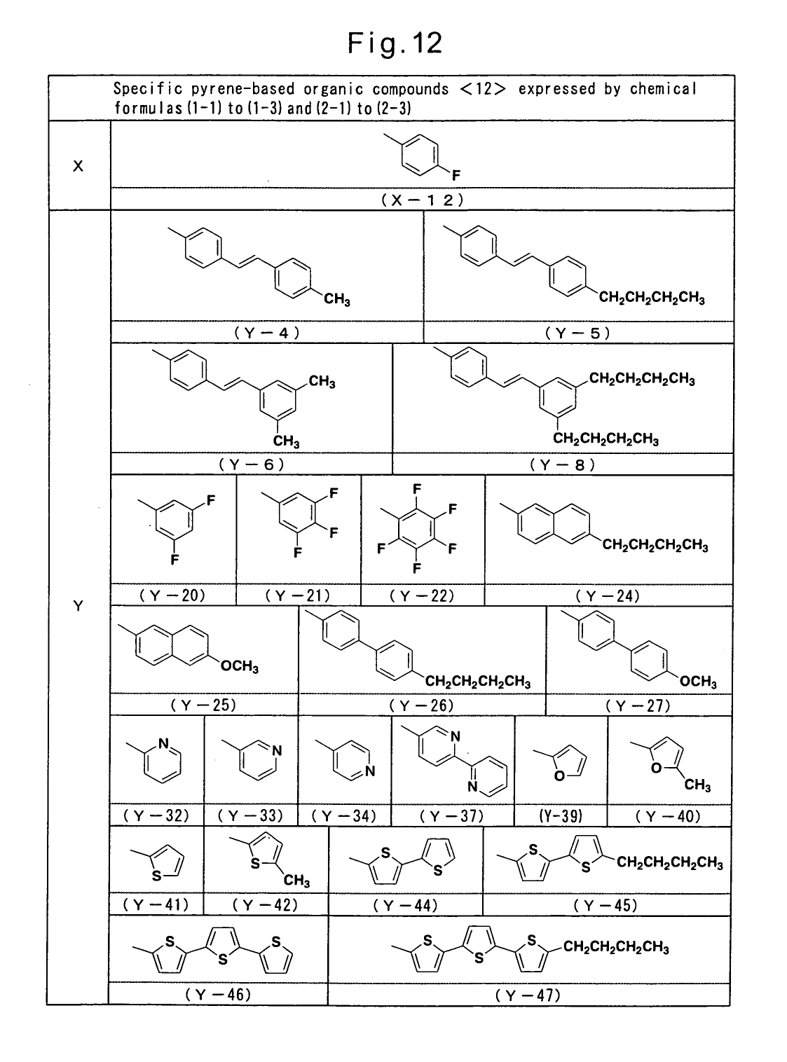

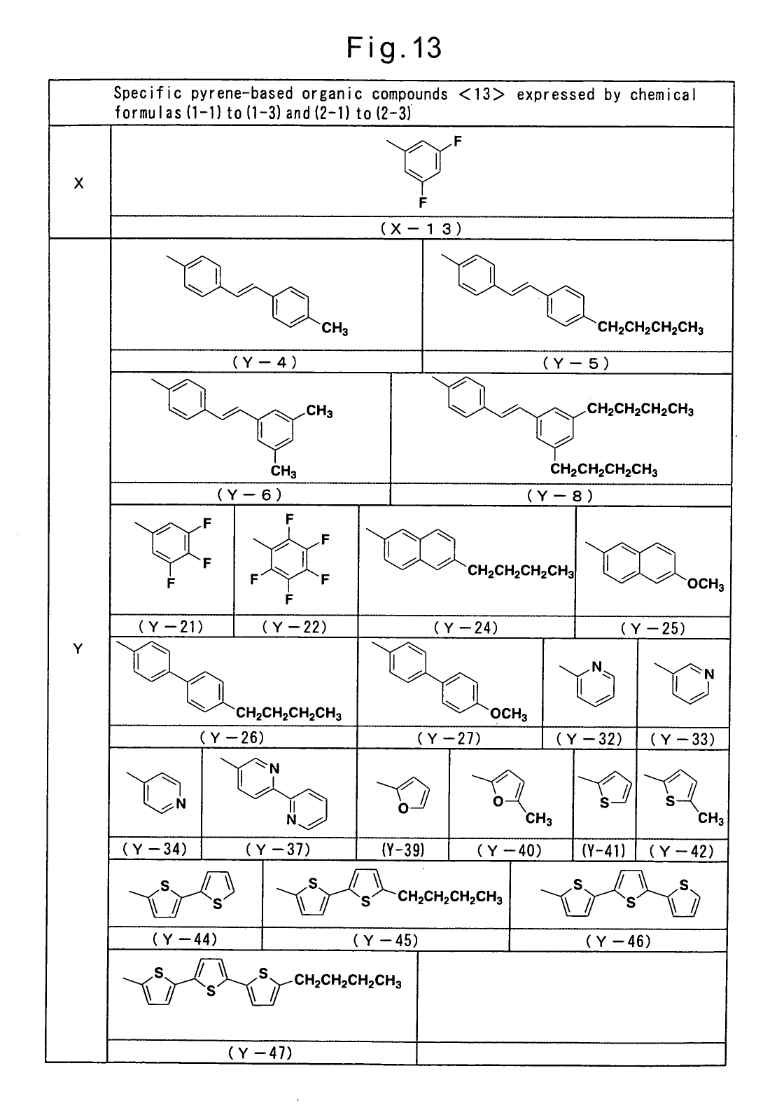

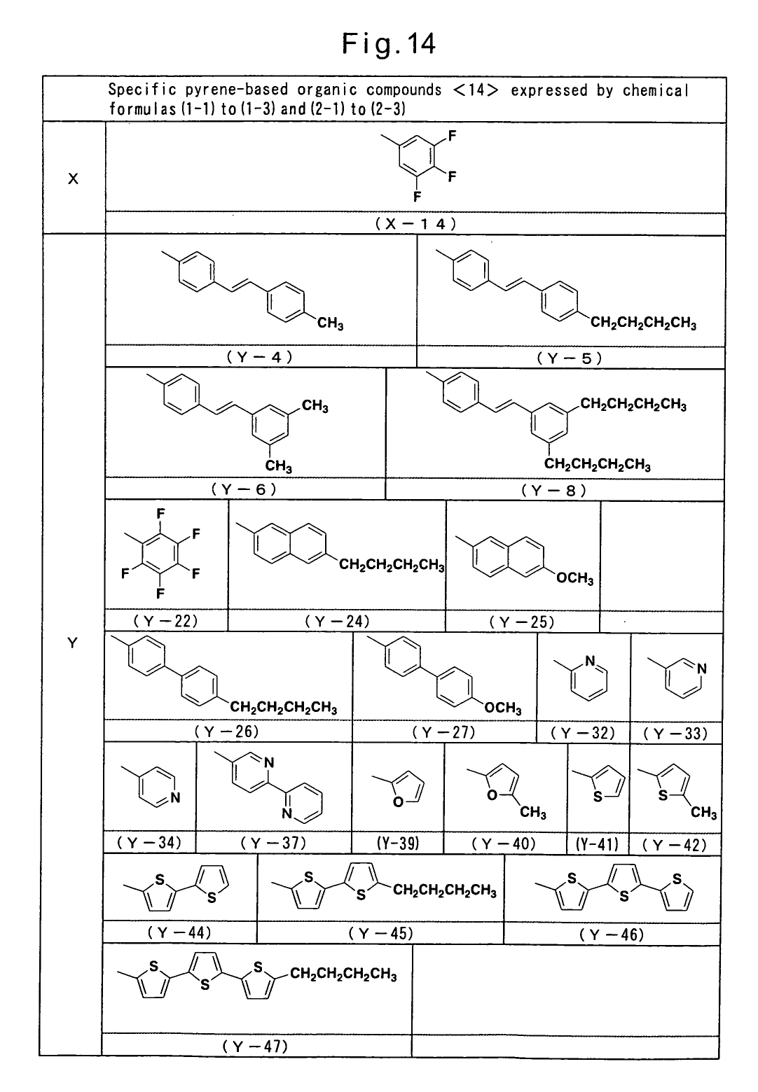

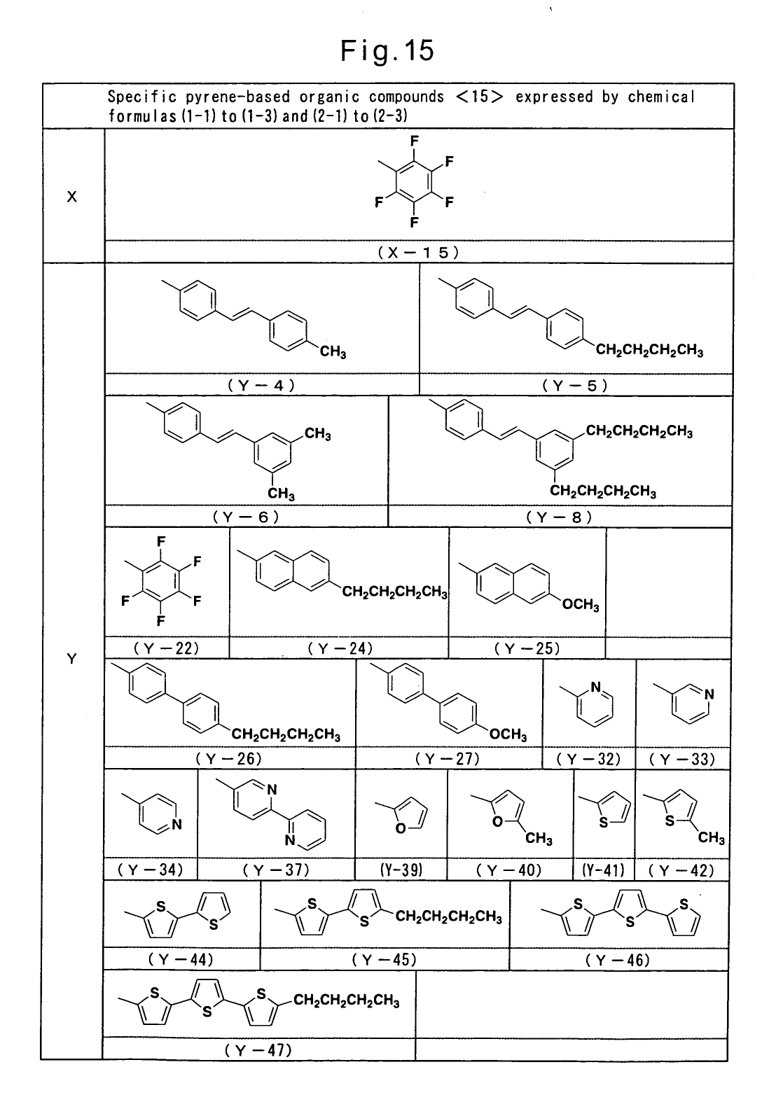

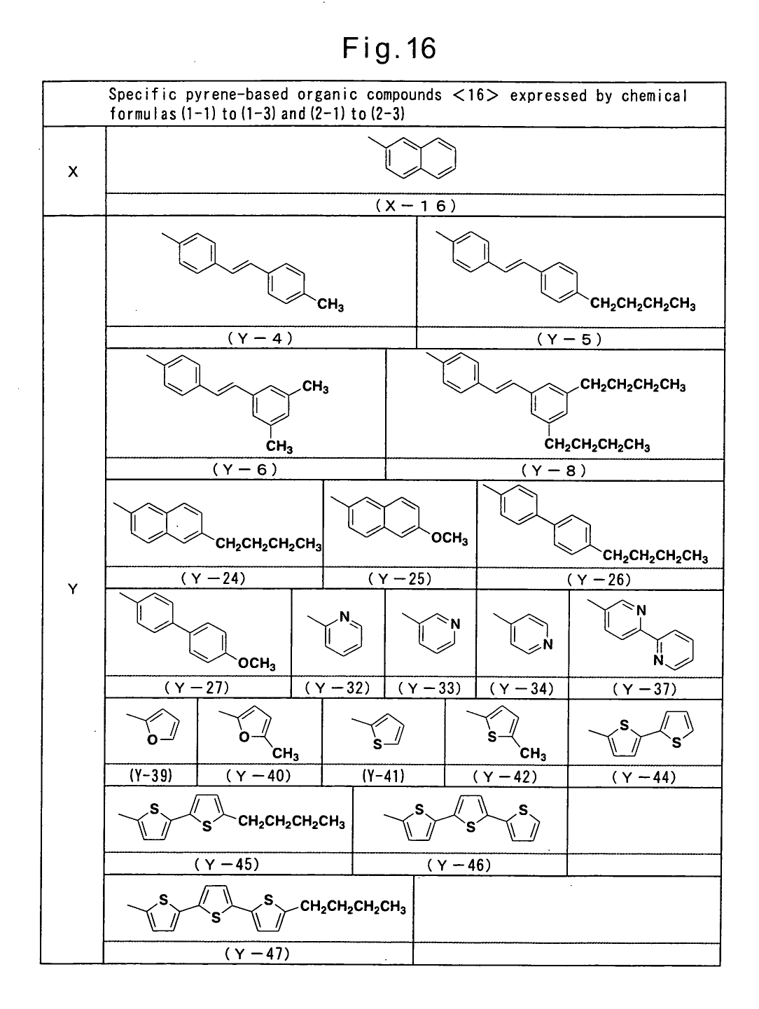

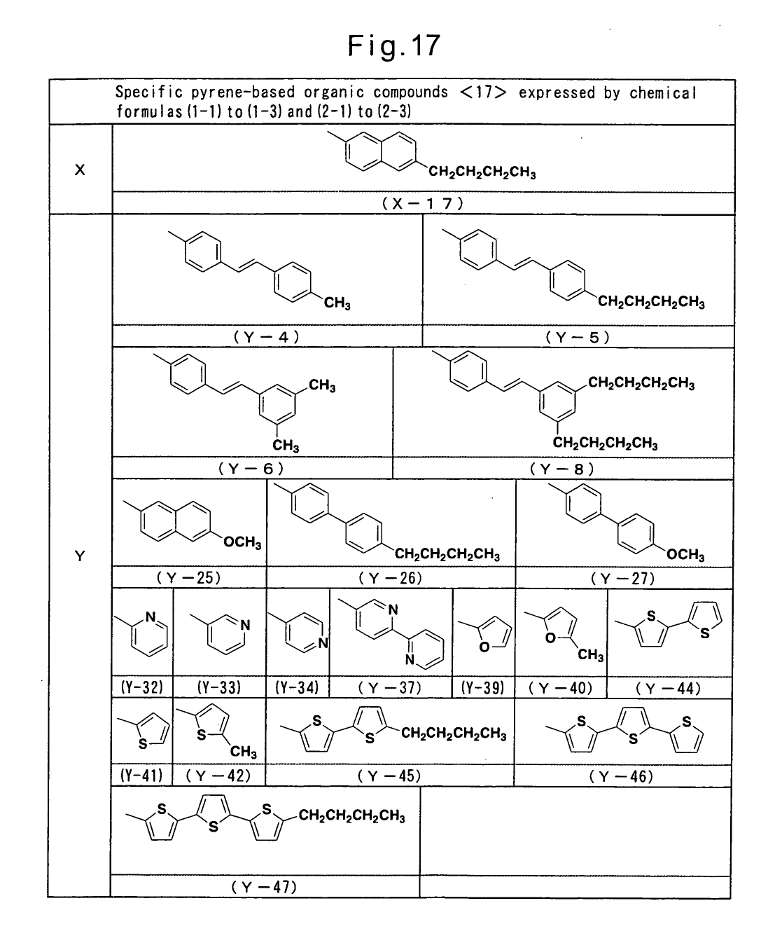

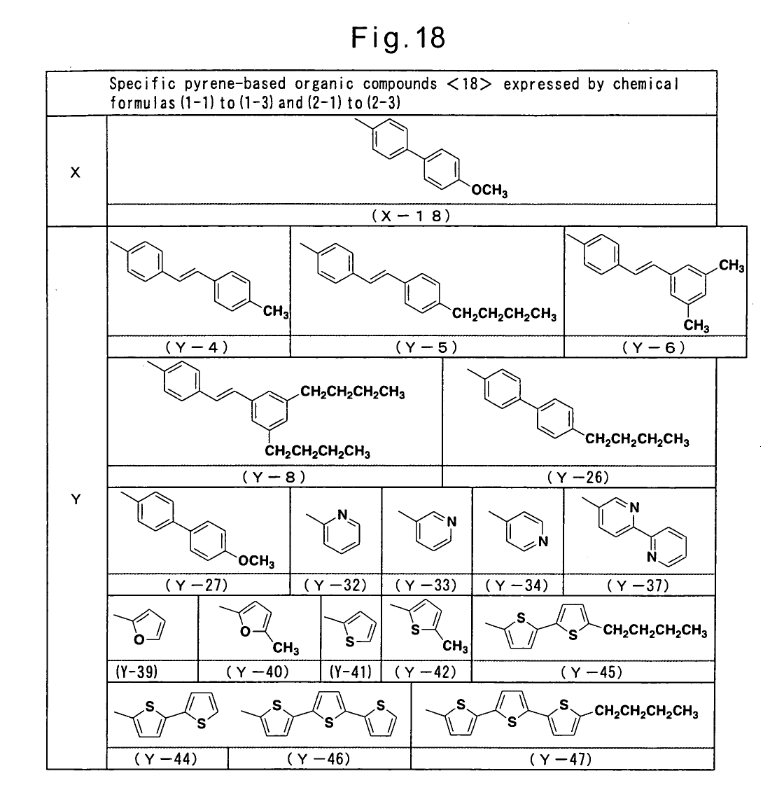

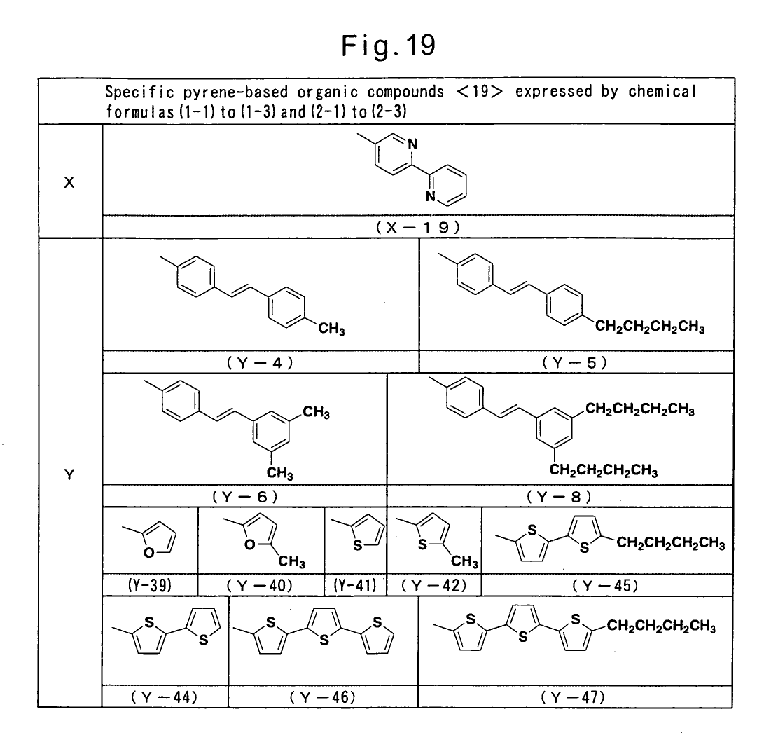

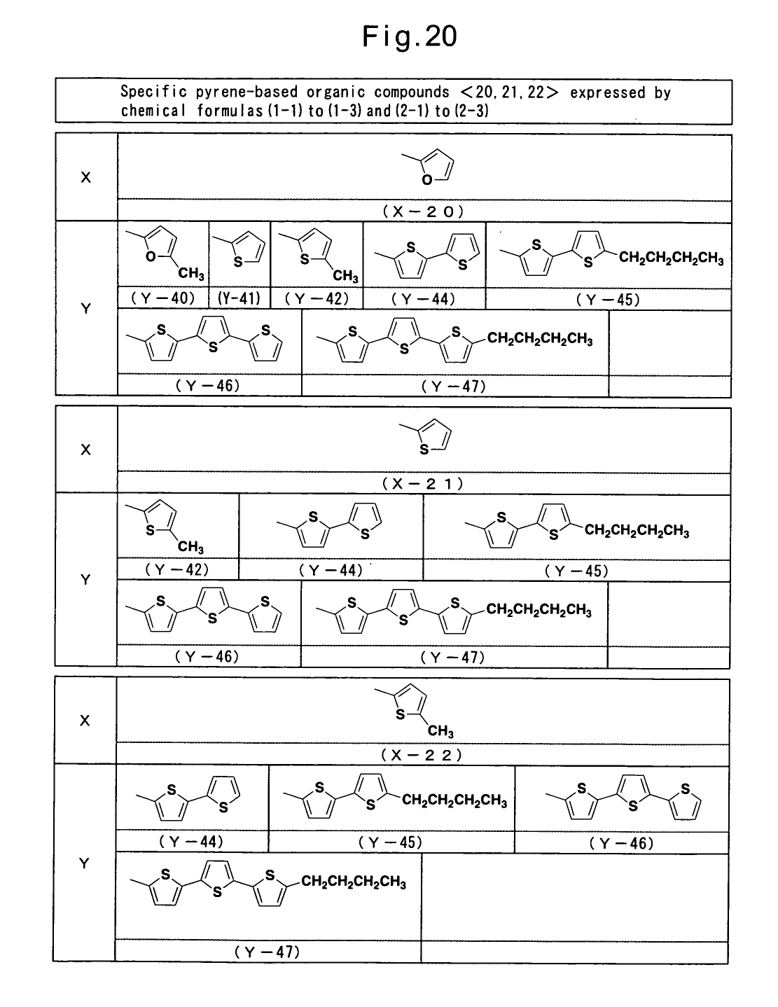

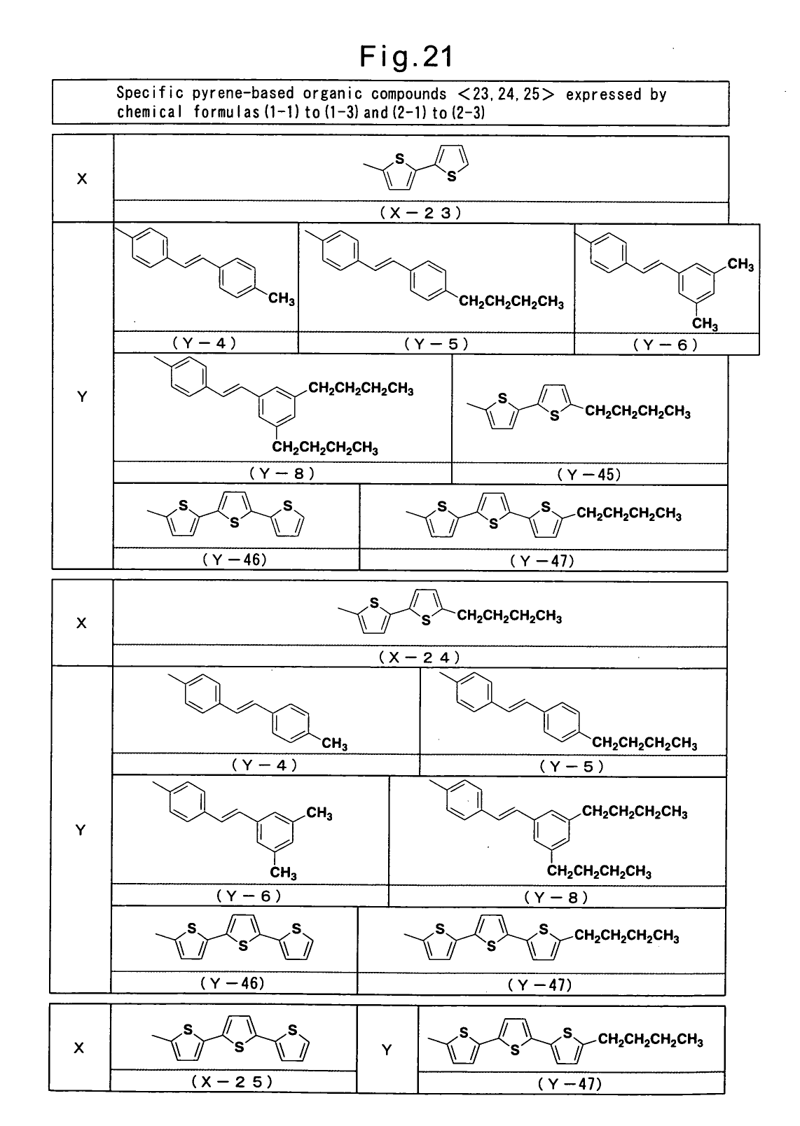

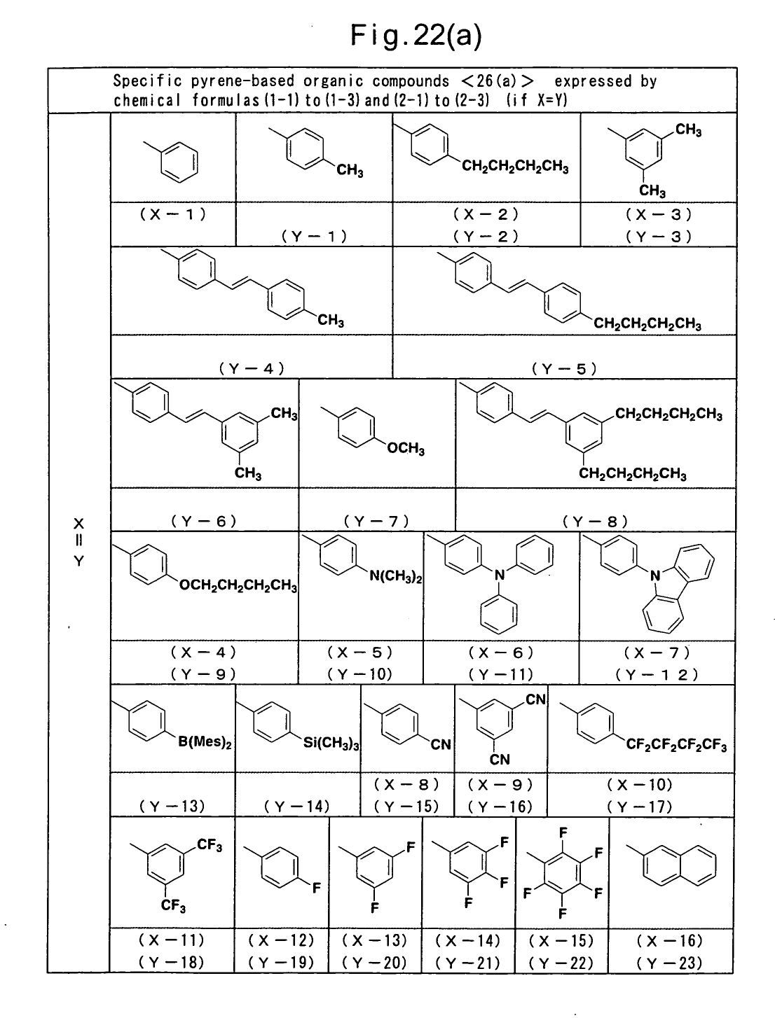

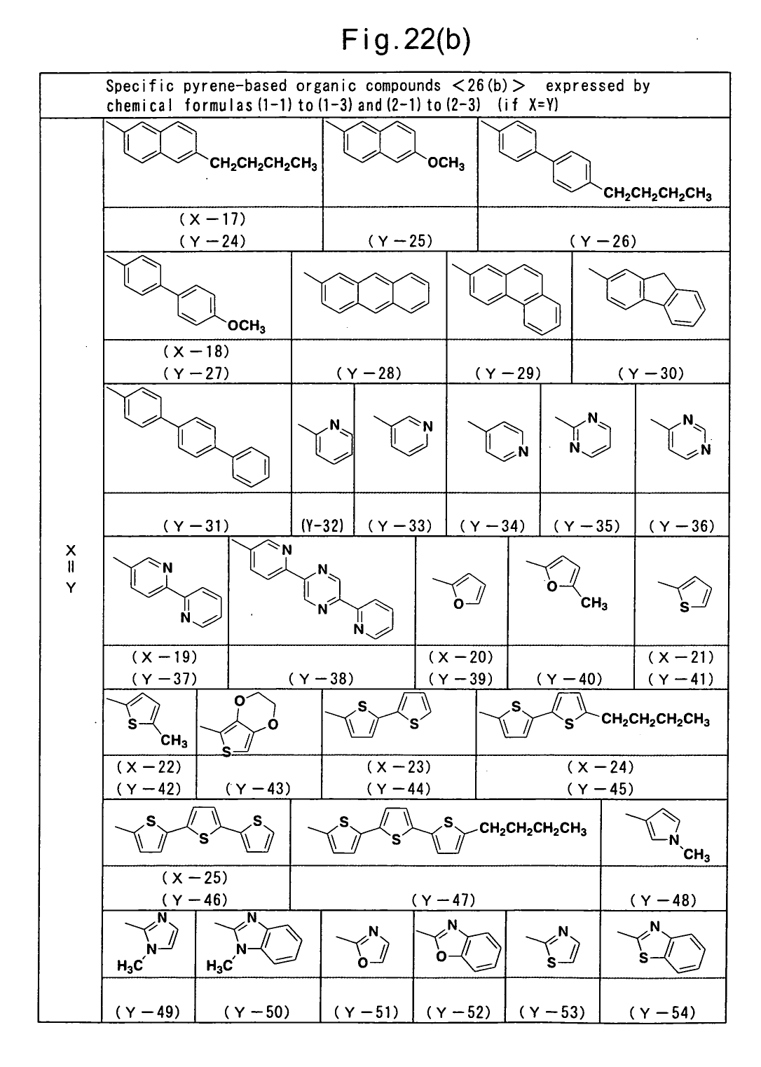

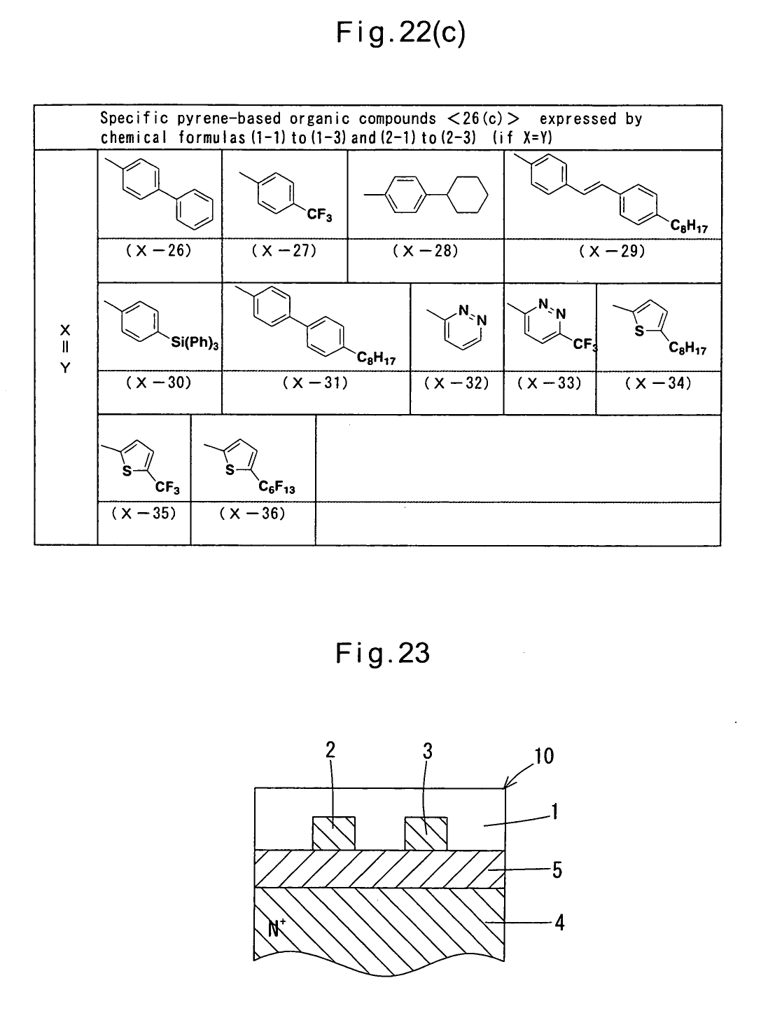

FIGS. 1 to 22 show the specific examples <1> to <26> of X and Y. In FIGS. 1 to 22, only X and Y are shown. Specific examples <1> to <25> show examples Y in combination with specific examples of X (i.e. (X-1) to (X-25). In specific examples <1> to <25>, X is not identical to Y. That is, these examples show asymmetrical pyrene-based organic compounds. Compounds wherein X is identical to Y, i.e. symmetrical pyrene-based organic compounds are shown as specific examples <26>. The groups represented by X and Y in each compound are denoted by different numerals even if they are identical to each other. To any of FIGS. 1-22 and specific examples <1> to <26> that extends more than one page, symbols (a), (b) and (c) are affixed to the main number for the respective pages.

Each of the compounds expressed by the formulas (1) and (2) should preferably have a molecular weight of not less than 500, more preferably not less than 800, and not more than 5000, more preferably not more than 3000. By determining the molecular weight within this range, it is possible to impart stability to the compounds.

Each of X and Y, including its substituents, should preferably has a molecular weight of not more than 5000, more preferably not more than 3000, further preferably not more than 1000, and especially preferably not more than 200. By determining the molecular weight within this range, it is possible to impart stability to the compounds.

The compounds shown by the formulas (1) and (2) can be used as transistor materials. A transistor material made of any of the compounds represented by the formulas (1) and (2) has a high carrier mobility and high light-emitting properties, so that this material can be used especially as a light-emitting transistor material.

Among compounds represented by the formulas (1) and (2), compounds represented by the below formulas (3) to (5) are novel compounds that is useful as transistor materials, particularly light-emitting transistor materials. Because these compounds can also be used as light-emitting pigments too, they can be used not only as transistor materials but as organic EL pigments, dye lasers, photoelectric conversion materials, and medical diagnostic reagents.

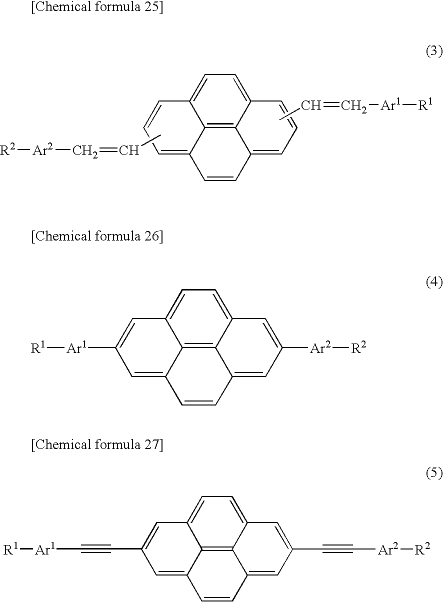

In the formulas (3), (4) and (5), Ar¹ and Ar² represent, independently of each other, aromatic hydrocarbon groups other than a pyrene ring or aromatic heterocyclic groups. R¹ and R² represent, independently of each other, halogen atoms, alkyl groups which may have a substituent having a carbon number of 2-20 or alkoxyl groups having a carbon number of 1-20. Each of Ar¹ and Ar² has one or more of R¹ or R². The abovementioned substituent is a group selected from aromatic hydrocarbon groups having a carbon number of 6-36, aromatic heterocyclic groups having a carbon number of 4-36, and halogen atoms.

### (How to Synthesize (3))

Description is now made of how to synthesize the compound represented by the formula (3).

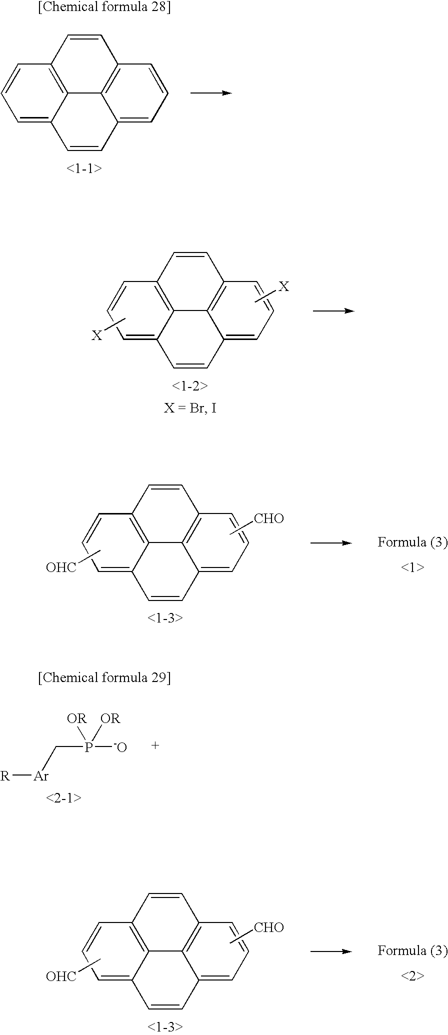

Ordinarily, the compound represented by the formula (3) can be produced according to the above reaction formula <1>.

First, the compound represented by the formula <1-2> can be produced using pyrene (formula <1-1>) by the method described in Bull. Chem. Soc. Jpn., 67, 172- (1994), J. Chem. Soc., Perkin Trans 1., 1622-(1972). That is, <1-2> is obtained from <1-1> by reaction in an organic solvent such as dimethylformamide, using bromine, N-bromosuccinimide (NBS), N-iodosuccinimide, iodine, sodium iodate.

The compound represented by the formula <1-3> can be synthesized using the compound represented by the formula <1-2> with the method described in European patent publication EP0964045. That is, <1-3> is obtained from <1-2> by reacting n-BuLi in THF (tetrahydrofuran) or ether at −78° C. to 0° C. for 30 minutes to 5 hours, then adding dimethylformamide, and hydrolyzing with acid.

Ordinarily, the compound represented by the formula (3) can be synthesized from the compound represented by the formula <1-3> using the Horner-Wadsworth-Emmons method. That is, as shown by the reaction formula <2>, it is obtainable by reacting a corresponding phosphate (formula <2-1>) with the compound represented by the formula <1-3> in an organic solvent in the presence of a base. The base is preferably sodium hydride, t-butoxy potassium or sodium methoxide. The organic solvent is preferably dimethylformamide, THF, toluene or benzene. The reaction temperature is preferably 0° C. to 110° C., though it varies with the organic solvent used. Further, crown ether may be used as an additive.

### (How to Synthesize (4) and (5))

Description is now made of how to synthesize the compounds represented by the formulas (4) and (5).

First, an ordinary method for synthesizing the compound represented by the formula (1-3-1) is described.

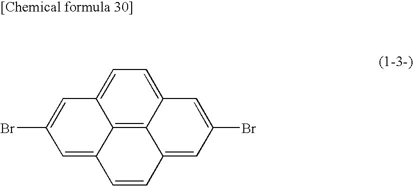

The compound represented by the formula (1-3-1) (2,7-dibromopyrene) can be synthesized by methods described in publications. That is, it can be synthesized by obtaining corresponding metacyclophane dione, by coupling 1,3-(bromomethyl)benzene, p-toluene sulfonylmethyl and isocyanide (Publication: Tetrahedron Letters, 1982, 23, 5335-5338), decarbonylating it, thereby converting it to metacyclophane (Publication: Euro. J. Org. Chem. 2001, 2487-2499), and acting bromine thereon (Publication: Chem. Ber. 1984, 117, 260-276; Synth. Commun. 1988, 18, 2207-2209; J. Org. Chem. 1986, 51, 2847-2848). Specifically, it can be produced by the method represented by the following reaction formula <1-4>.

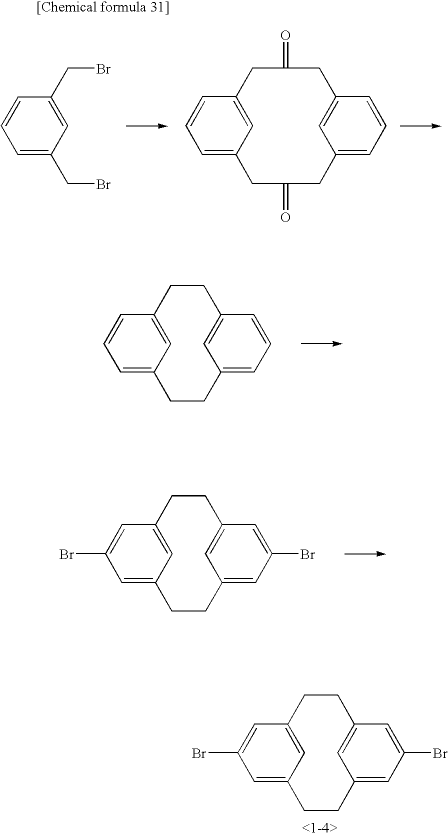

An ordinary method for synthesizing the compound represented by the formula (4) is now described.

The compound represented by the formula (4) can be produced from the compound represented by the formula <1-3-1> as a starting material according to the formula <2-3>.

That is, this compound is obtained by reacting the compound represented by the formula <2-2> and the compound represented by the formula (1-3-1) using a palladium catalyst (such as Pd(PPh₃)₄ or PdCl₂(PPh₃)₂), an Ni catalyst and a base (such as K₂CO₃, Na₂CO₃ or Et₃N) in an organic solvent such as toluene, dioxane or THF in the presence of an inert gas (such as N₂ or Ar) at a temperature between room temperature and the boiling point of the solvent.

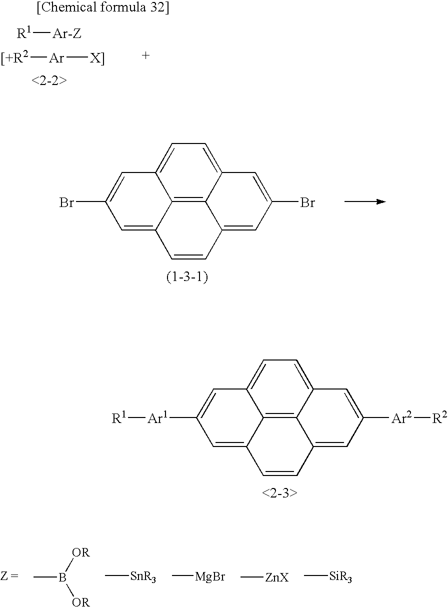

Alternatively, this compound can be produced according to the formula <2-4>.

That is, it is obtained by reacting the compound represented by the formula (1-3-2), which can be synthesized by the method described in Publication Chem. Commun., 2005, 2172-2174, and the compound represented by the formula <2-2> using a palladium catalyst (such as Pd(PPh₃)₄ or PdCl₂(PPh₃)₂), an Ni catalyst and a base (such as K₂CO₃, Na₂CO₃ or Et₃N) in an organic solvent such as toluene, dioxane or THF in the presence of an inert gas (such as N₂ or Ar) at a temperature between room temperature and the boiling point of the solvent.

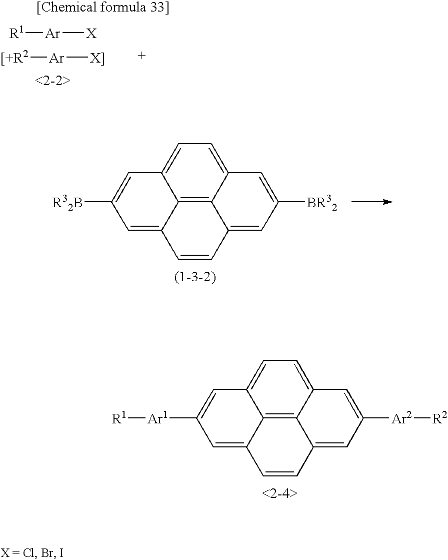

Ar in the reaction formulas <2-3> and <2-4> is identical to Ar¹ or Ar² shown in the formula (4). Also, R¹ and R² are identical to Ar¹ or Ar² shown in the formula (4).

An ordinary method for synthesizing the compound represented by the formula (5) is now described.

Ordinarily, the compound represented by the formula (5) can be produced according to the below formula <2-6>.

That is, this compound is obtained by reacting the compound represented by the formula (1-3-1) and the compound represented by the formula <2-5> using a palladium catalyst (such as Pd(PPh₃)₄ or PdCl₂(PPh₃)₂), and copper iodide in a solvent such as diisopropylamine, diethylamine or pyridine in the presence of an inert gas (such as N₂ or Ar) at a temperature between room temperature and the boiling point of the solvent.

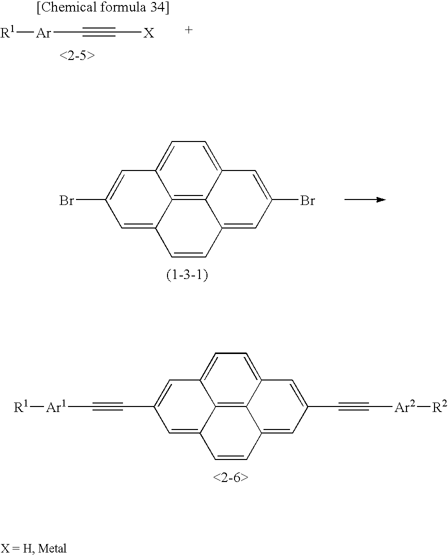

Description is now made of a light-emitting transistor device using the above-mentioned pyrene-based compound.

The light-emitting transistor device may be one having a basic structure of a field effect transistor (FET) as shown in FIG. 23.

This light-emitting transistor device 10 comprises a light-emitting layer 1 which is capable of transporting holes and electrons as carriers, which emits light by recombination of the holes and the electrons, and which contains the above-mentioned pyrene-based compound as a main component; a hole injecting electrode for injecting holes into this light-emitting layer 1, i.e., what is called a source electrode 2; an electron injecting electrode for injecting electrons into the light-emitting layer, i.e., what is called a drain electrode 3; and a gate electrode 4 which is provided opposite to the source electrode 2 and the drain electrode 3 and is made of an N⁺ silicon substrate to control the distribution of the carriers in the light-emitting layer 1. The gate electrode 4 may be made of an electroconductive layer comprising an impurity diffusion layer formed on the surface of the silicon substrate.

Specifically, as shown in FIG. 23, an insulating film 5 made of silicon oxide or the like is formed on the gate electrode 4, and the source electrode and the drain electrode 3 are formed thereon at an interval. The light-emitting layer 1 is formed to cover the source electrode 2 and the drain electrode 3 and to be disposed between the two electrodes.

In order to improve the carrier mobility, the silicon substrate may be treated after forming the insulating film 5 or after forming the source electrode 2 and the drain electrode 3. There are two types of such treatments, i.e. surface treatment and control of the substrate temperature.

The surface treatment is a method in which after forming the insulating film 5 or after forming the source electrode 2 and the drain electrode 3, the surface is treated with UV ozone and a surface treating agent is applied thereto. As the surface treating agent, a known surface treating agent may used, such as HMDS (hexamethyldisilazane) or OTS (octyltrichlorosilane). After applying the surface treating agent, its residue is removed, and a compound used for the light-emitting layer is deposited under vacuum.

The temperature control of the silicon substrate is a method in which a compound used for the light-emitting layer is deposited under vacuum on the substrate after forming the insulating film 5 or after forming the source electrode 2 and the drain electrode 3 at a constant temperature by applying heat under vacuum. The substrate is preferably kept at a temperature within the range of 40 to 80° C. Both the surface treatment and the temperature control may be carried out.

In order for the above-mentioned device to exhibit the function of the light-emitting transistor, it is preferred that the difference between the HOMO energy level and the LUMO energy level of the organic fluorescent substance which constitutes the light-emitting layer 1, in particular, the pyrene-based compound as the main component thereof, the carrier mobility thereof, the on/off ratio, or the luminous efficiency thereof satisfies a predetermined range.

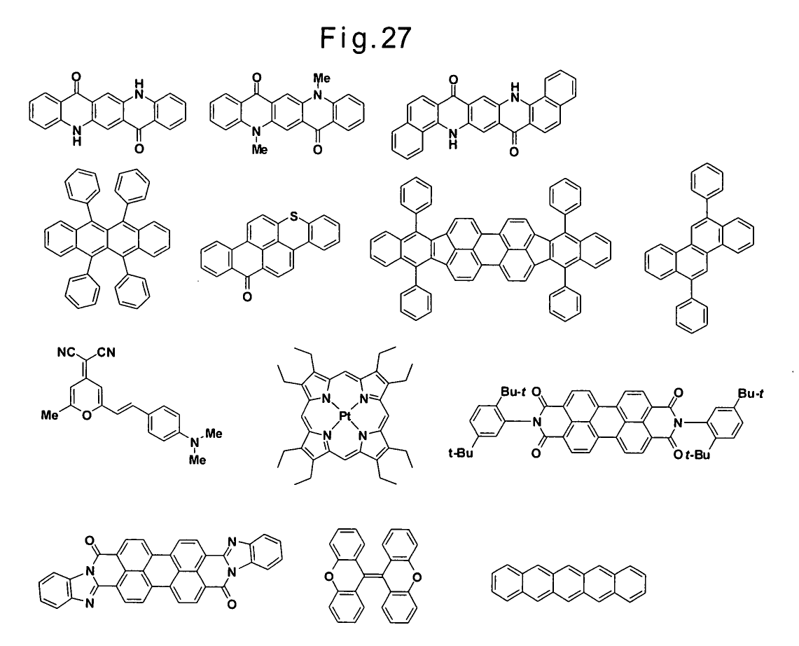

The light-emitting layer 1 may be made of one or more than one of the pyrene-based compounds according to the present invention. By adding a sub-component having a different wavelength, such as an organic fluorescent substance, it is possible to improve the respective functions. If an organic fluorescent substance having a different wavelength is combined, it may be doped in the amount of not less than 0.1%, preferably not less than 0.5%, and not more than 20%, preferably not more than 10% of the pyrene-based compound as the main component (or of the total content of a plurality of kinds of the pyrene-based compounds according to the present invention). The organic fluorescent substance having a different wavelength may be one of the compounds shown in FIG. 27 but is not limited thereto.

First, the difference between the HOMO energy level and the LUMO energy level is preferably as small as possible so that the electrons can move more easily, and thus the light emission and the semiconductivity (that is, the conductivity of electrons or holes in one direction) can be generated more easily. Specifically, the difference is preferably 5 eV or less, more preferably 3 eV or less, even more preferably 2.7 eV or less. Because the smaller this difference, the better the results, the lower limit of this difference is 0 eV.

The carrier mobility is preferably as high as possible for improved semi-conductivity. Specifically, the carrier mobility is preferably 1.0×10⁻⁵ cm²/V·s or more, more preferably 4.0×10⁻⁵ cm²/V·s or more, even more preferably 1.0×10⁻⁴ cm²/V·s or more. The upper limit of the carrier mobility is not particularly limited, and it is sufficient if the upper limit is about 1 cm²/V·S.

The on/off ratio is a ratio of the maximum Id (Ion) when the gate voltage is [−100 V] to the minimum Id (Ioff) when the gate voltage is [0 V], which gives the off position, with the drain voltage at [−100 V]. It is possible to determine that the higher this ratio, the higher the semiconductivity. This ratio is preferably not less than 10², more preferably not less than 10³.

The above-mentioned luminous efficiency means the ratio of light generated by the injection of photons or electrons. The ratio of emitted optical energy to injected optical energy is defined as the PL luminous efficiency (or PL quantum efficiency), and the ratio of the number of emitted photons to the number of injected electrons is defined as the EL luminous efficiency (or the EL quantum efficiency).

Injected and excited electrons emit light by recombining with holes. This recombination does not necessarily occur with a probability of 100%. Therefore, when organic compounds which each constitute the light-emitting layer 1 are compared with each other, the EL luminous efficiencies are compared, thereby making it possible to compare the ratios of the emitted optical energy amount to injected optical energy, and compare synergetic effects about the ratio of the recombination of electrons and holes. Incidentally, by comparing the PL luminous efficiencies, the ratios of the emitted optical energy amount to injected optical energy can be compared. Thus, by comparing both the PL luminous efficiencies and the EL luminous efficiencies and combining the results, it is possible to compare the ratios of the recombination of electrons and holes.

For the PL luminous efficiency, the degree of light emission is preferably as high as possible. The PL luminous efficiency is preferably 20% or more, more preferably 30% or more. The upper limit of the PL luminous efficiency is 100%. The lower limit of the PL luminous efficiency is preferably 3% and more preferably 5%.

For the EL luminous efficiency, the degree of light emission is preferably as high as possible. The EL luminous efficiency is preferably 1×10⁻³% or more, more preferably 5×10⁻³% or more. The upper limit of the EL luminous efficiency is 100%.

The light-emitting transistor device 10 is characterized by the wavelength of emitted light besides the above. This wavelength is in a visible ray range. The device has a wavelength varied in accordance with the kind of the organic fluorescent substance used, in particular, the pyrene-based compound. When organic fluorescent substances having different wavelengths are combined with each other, various colors can be produced. For this reason, about the wavelength of emitted light, the wavelength itself exhibits a characteristic.

The light-emitting transistor device 10 is characterized by light emission. Thus, the device preferably has a luminous brightness to a certain extent. This luminous brightness is defined as the light emission amount corresponding to the brightness of an object felt by a person when the person watches the object. This luminous brightness is preferably as high as possible when measured by a photo-counter. The luminous brightness is preferably 1×10⁴ CPS (count per sec) or more, more preferably 1×10⁵ CPS or more, even more preferably 1×10⁶ CPS or more.

The light-emitting layer 1 is formed by depositing an organic fluorescent substance or the like that constitute the light-emitting layer 1 (or co-depositing a plurality of such substances). It is sufficient if the film thickness of this light-emitting layer is at least about 70 nm.

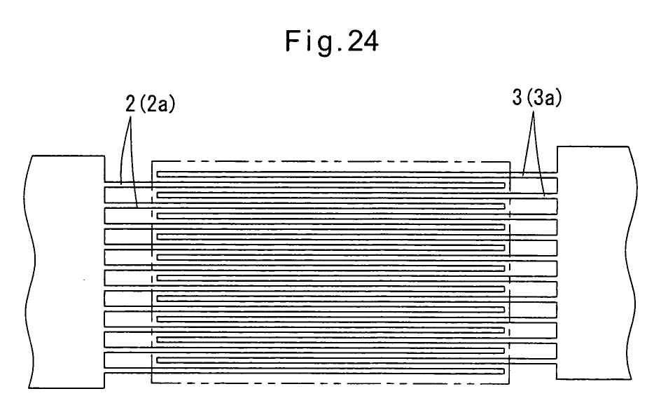

The source electrode 2 and the drain electrode 3 are electrodes for injecting holes and electrons into the light-emitting layer 1, and are made of gold (Au), magnesium-gold alloy (MgAu), or the like. The electrodes are formed so as to face each other at a very small interval of, for example, 0.4 to 50 μm. Specifically, for example, as shown in FIG. 24, the source electrode 2 and the drain electrode 3 are formed to have comb tooth shaped regions 2a and 3a, respectively, which are each made of a plurality of comb teeth. The comb teeth which constitute the comb tooth shaped region 2a of the source electrode 2 and the comb teeth which constitute the comb tooth shaped region 3a of the drain electrode 3 are alternately arranged at predetermined intervals, whereby the light-emitting transistor device 10 can exhibit the function thereof more effectively.

At this time, the interval between the source electrode 2 and the drain electrode 3, that is, the interval between the comb tooth shaped region 2a and the comb tooth shaped region 3a is preferably 50 μm or less, more preferably 3 μm or less, even more preferably 1 μm or less. If the interval is more than 50 μm, sufficient semiconductivity cannot be exhibited.

By applying a voltage to the source electrode 2 and the drain electrode 3 in the light-emitting transistor device 10, holes and electrons are shifted inside the device and they are recombined in the light-emitting layer 1, whereby light can be emitted. At this time, the amounts of the holes and the electrons shifted between the two electrodes across the light-emitting layer 1 depend on the voltage applied to the gate electrode 4. Accordingly, by controlling the voltage applied to the gate electrode 4 and its change, it is possible to control the state of electric conduction between the source electrode 2 and the drain electrode 3. Because this light-emitting transistor device 10 undergoes P-type driving, a negative voltage for the source electrode 2 is applied to the drain electrode 3 and a negative voltage for the source electrode 2 is applied to the gate electrode 4.

Specifically, by applying a negative voltage for the source electrode 2 to the gate electrode 4, holes in the light-emitting layer 1 are attracted toward the gate electrode 4, so that the density of holes in the vicinity of the surface of the insulating film 5 increases. By suitably adjusting the voltage between the source electrode 2 and the drain electrode 3, holes are injected from the source electrode 2 into the light-emitting layer 1 according to the intensity of the controlled voltage applied to the gate electrode 4, so that electrons are injected from the drain electrode 3 into the light-emitting layer 1. In other words, the source electrode 2 functions as a hole injecting electrode, and the drain electrode 3 functions as an electron injecting electrode. In this way, in the light-emitting layer 1, the holes and the electrons are recombined, and light is emitted following this recombination. This light emission state can be turned on or off or the luminous intensity can be varied by changing the controlled voltage applied to the gate electrode 4.

The theory of such recombination of holes and electrons can be described as follows:

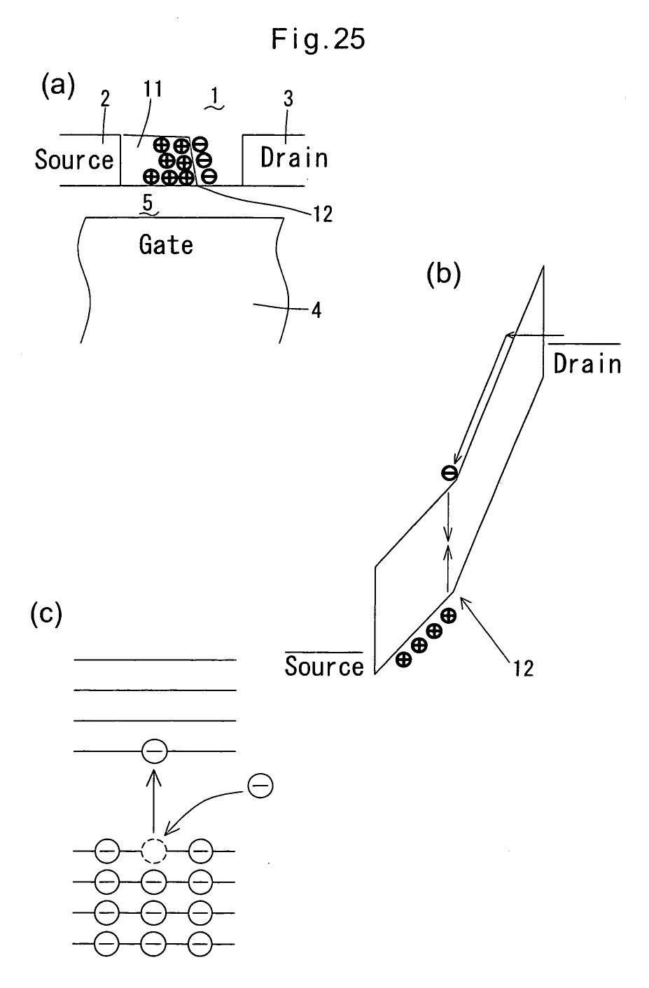

When a negative voltage for the source electrode 2 is applied to the gate electrode 4, in the light-emitting layer 1, as illustrated in FIG. 25(a), channel 11 of holes are formed near the interface of the insulating film 2 so that a pinch-off point 12 thereof forms in the vicinity of the drain electrode 3. A high electric field is then formed between the pinch-off point 12 and the drain electrode 3, so that as shown in FIG. 25(b), the energy band is significantly bent. This produces an FN (Fowler-Nordheim) tunnel effect in which electrons in the drain electrode 3 penetrate through the potential barrier between the drain electrode 3 and the light-emitting layer 1, so that the electrons are injected into the light-emitting layer 1 and recombined with the holes.

The recombination of holes and electrons can also be described on the basis of the following theory besides the FN tunnel effect. As shown in FIG. 25(c), electrons at the HOMO energy level of the organic fluorescent substance in the light-emitting layer 1 are excited to the LUMO level thereof by a high electric field. The excited electrons are recombined with holes in the light-emitting layer 1. At the same time, electrons are injected from the drain electrode 3 to the HOMO energy level, which is now empty due to the excitation to the LUMO energy level, so that the empty level is filled.

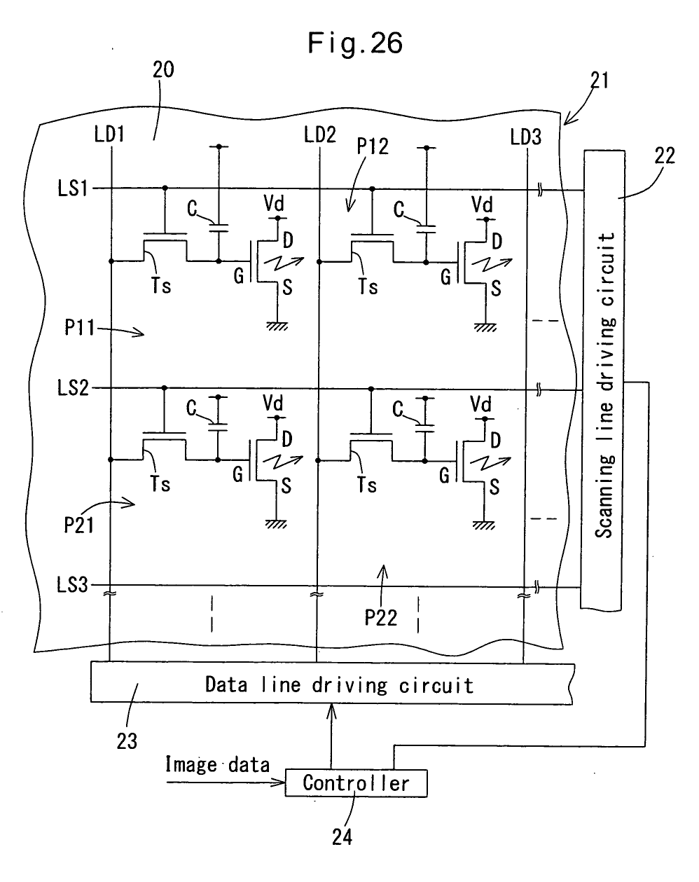

A plurality of such light-emitting transistor devices 10 are two-dimensionally arranged on a substrate 20 to form a display device 21. FIG. 26 shows an electric circuit diagram of this display device 21. Specifically, in this display device 21, light-emitting transistor devices 10 as described above are each arranged in one of pixels P11, P12, . . . , P21, P22, . . . , . . . , which are arranged in a matrix form. The light-emitting transistor devices 10 in these pixels are selectively caused to emit light and further the luminous intensity (brightness) of the light-emitting transistor device 10 in each of the pixels is controlled, whereby two-dimensional display can be attained. The substrate 20 may be, for example, a silicon substrate integrated with the gate electrode 4. In other words, the gate electrode 4 may be made of an electroconductive layer which is an impurity diffusion layer wherein a pattern is formed in a surface of a silicon substrate. As the substrate 20, a glass substrate may be used.

Since each of the light-emitting transistor devices 10 undergoes P-type driving, a bias voltage V_{d} (<0) is given to its drain electrode 3(D) with the source electrode 2(S) kept at the ground voltage (═O). To its gate electrode 4(G), a selecting transistor Ts for selecting a pixel and a capacitor C for storing data are connected in parallel.

The selecting transistors Ts in each row of the pixels P11, P12, . . . , . . . , P21, P22, . . . , have their gates connected to a common one of the scanning line LS1, LS2, . . . , . . . . The selecting transistors Ts in each column of the pixels P11, P21, . . . , . . . , P12, P22, . . . , . . . are connected to a common one of the data lines LD1, LD2 . . . on their side opposite to the respective light-emitting transistor devices 10.

From a scanning line driving circuit 22 controlled by a controller 24, scanning driving signals for selecting the pixels P11, P12, . . . , . . . P21, P22, . . . , . . . in the respective rows circularly and successively (selecting the plurality of pixels in each row at a time) are given to the scanning lines LS1, LS2, . . . , . . . . In other words, the scanning line driving circuit 22 makes it possible to specify each of the rows successively as a selected row and make the selecting transistors Ts of the plurality of pixels in the selected row electrically conductive at a time, thereby generating a scanning driving signal for cutting off the selecting transistors Ts of the plurality of pixels in the non-selected rows at a time.

On the other hand, signals from a data line driving circuit 23 are inputted into the data lines LD1, LD2, . . . , . . . . Control signals corresponding to image data are inputted from the controller 24 into this data line driving circuit 23. At a timing when the pixels in each of the rows are collectively selected by the scanning line driving circuit 22, the data line driving circuit 23 supplies light emission controlling signals, which correspond to the light emission gradations of the individual pixels in the selected row, to the data lines LD1, LD2, . . . , . . . in parallel.

In this way, in the individual pixels in the selected row, the light emission controlling signals are given to the gate electrodes 4(G) through the selecting transistors Ts. Thus, the light-emitting transistor devices 10 in the pixels emit light having gradations corresponding to the light emission controlling signals (or stop the light emission). Since the light emission controlling signals are kept in the capacitor C, the electric potentials of the gate electrodes 4(G) are kept even after the selected row selected by the scanning line driving circuit 22 is shifted to a different row. As a result, the light emission states of the light-emitting transistor devices 10 are kept. Thus, two-dimensional display can be attained.

### EXAMPLES

The present invention is more specifically described by way of examples. The present invention is not limited to these examples but encompasses any modification that is within the purview of the present invention. First, methods of producing pyrene-based compounds are described.

### (Synthesis of Raw Intermediate Material)

### (Production Example 1 of Raw Intermediate Material)[Production of Dibromopyrene]

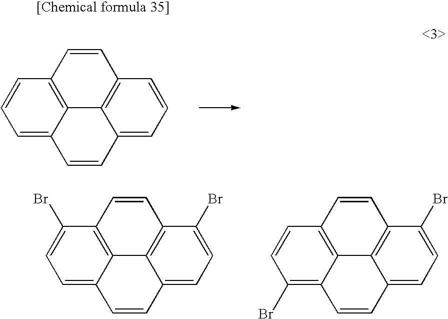

A 200 ml four-necked flask was fitted with a dropping funnel and a three-way cock connected to a nitrogen line, and was purged with nitrogen. Then, 5.00 g of pyrene (reagent made by Tokyo Chemical Industry Co., Ltd., purity: 97%) and 30 ml of DMF (reagent made by Junsei Chemical Co., Ltd.) were added, the flask was again purged with nitrogen, and the system was stirred at room temperature. A solution in which 8.81 g of N-bromosuccinimide (NBS, a reagent made by Tokyo Chemical Industry Co., Ltd., purity: 98%) was dissolved in 25 ml of DMF, as identified above, was dropwise added for 20 minutes, and after the end of dropping, the mixture was further stirred at room temperature for 9 hours. A yellow solid produced by the reaction was collected by suction filtration, and washed by suspension in ethanol (reagent made by Junsei Chemical Co., Ltd.) to obtain a crude product.

The resulting solid was recrystallized three times using toluene (reagent made by Junsei Chemical Co., Ltd.) to remove monobromopyrene. After the three-time recrystallization, a white solid was obtained which was a mixture of mainly 1,6-dibromopyrene and 1,8-dibromopyrene.

From ¹H-NMR of the collected product, it was confirmed that the mixture ratio of 1,6-dibromopyrene and 1,8-dibromopyrene was 1:1. The LC purity of the collected product was 93% as the mixture of dibromopyrenes, with 1,3,6-tribromopyrene being 7%. The yielded amount was 4.48 g and the yield was 50.3%.

⁻¹H NMR (CDCl₃, 400 MHz)

1,8-dibromopyrene . . . δ8.53 (s, 2H), 8.28 (d, 2H, J=8.40), 8.05 (d, 2H, J=8.00), 8.04 (s, 2H)

6-dibromopyrene . . . δ8.47 (d, 2H, J=9.60), 8.27 (d, 2H, J=8.40), 8.13 (d, 2H, J=9.20), 8.06 (d, 2H, J=8.40)

### (Production Example 2 of Raw Intermediate Material) [Production of Diformylpyrene]

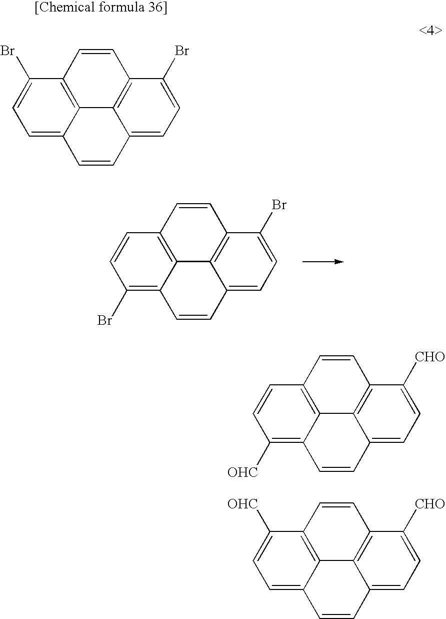

A 1000 ml four-necked flask was fitted with a dropping funnel, a three-way cock connected to a nitrogen line, a three-one stirrer motor, a glass shaft, a Teflon blade, and a low-temperature thermometer, heat drying and nitrogen purging were performed five times using a heat gun under reduced pressure to produce a nitrogen atmosphere in the system. Then, 15 g of a 1:1 mixture of 1,6-dibromopyrene and 1,8-dibromopyrene and 300 ml of dried THF (reagent made by Kanto Chemical Co., Inc.) were added, and the reactor was cooled to −70° C. in a dry ice-acetone bath. 80 ml of n-butyllithium (2.6M, a reagent made by Kanto Chemical Co., Inc.) was dropwise added for 20 minutes. After the end of dropping, the system was further stirred for two hours while keeping the temperature at −70° C. 32 ml of dried DMF (reagent made by Kanto Chemical Co., Inc.) was dropwise added for 10 minutes from the dropping funnel. During this process, heat build-up was confirmed and the internal temperature rose from −70° C. to −50° C. After the end of dropping of DMF, the system was further stirred for 30 minutes while cooling. Then, the cooling bath was removed, and the system was heated to room temperature and left to stand overnight. 1300 ml of 1N—HCl was slowly added, and a yellow solid deposited was collected by suction filtration to obtain a wet cake. The filtrate was separated and extracted with 300 ml of dichloromethane, condensed as it is without using a drying agent, and collected as the wet cake. The resulting cakes were combined, washed by suspension in ethanol, and dried under reduced pressure. It was confirmed that yellow crystals obtained from ¹H-NMR were a 1:1 mixture of 1,6-diformylpyrene and 1,8-diformylpyrene. The yielded amount was 6.78 g and the yield was 63.0%.

¹H-NMR (CDCl₃, 400 MHz)

6-diformylpyrene . . . δ10.82 (s, 2H), 9.60 (d, 2H, J=9.20), 8.55 (d, 2H, J=7.60), 8.43 (s, 2H, J=8.00), 8.36 (d, 2H, J=9.20)

1,8-diformylpyrene . . . δ10.87 (s, 2H), 9.62 (s, 2H), 8.57 (d, 2H, J=7.60), 8.43 (d, 2H, J=8.00), 8.26 (s, 2H)

### Production Examples

### Production Example 1

### Production of 1,6-diphenylpyrene (Compound 1), and 1,8-diphenylpyrene (Compound 2)

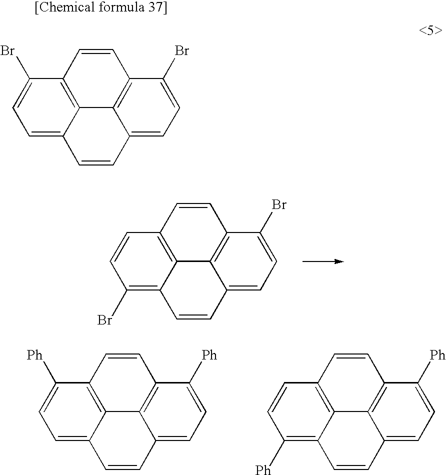

4.00 g of a 1:1 mixture of 1,6-dibromopyrene and 1,8-dibromopyrene, g (5.0 equivalent weight with respect to dibromopyrenes) of phenyl borate (reagent made by Tokyo Chemical Industry Co., Ltd., purity not identified), and 11.82 g of Na₂CO₃ (reagent made by Kanto Chemical Co., Inc.) were put into a 300 ml three-necked flask including a reflux condenser tube, a three-way cock connected to a nitrogen line, a thermometer and a rotor. 100 ml of toluene (reagent made by Junsei Chemical Co., Ltd.), 50 ml of ethanol (reagent made by Junsei Chemical Co., Ltd.), and 10 ml of desalted water were added and suspended. The operation of degassing the resulting suspension and returning the pressure to normal pressure with nitrogen was repeated five times. Then, the reaction mixture was bubbled with nitrogen for nitrogen purging in the system. 0.49 g of tetrakistriphenylphosphine palladium (reagent made by Tokyo Chemical Industry Co., Ltd.) was then added, and the system was stirred while heating at 80° C. for 10 hours in an oil bath.

Undissolved phenyl borate and inorganic salts were removed by sucking and filtering the reaction mixture. To the filtrate was added 100 ml of desalted water to separate the solution into phases. The organic phase was dehydrated with anhydrous magnesium sulfate and then condensed. Inorganic salts and palladium mixed therewith were removed by column chromatography (silica gel, CHCl₃) so as to yield a greenish yellow crude product. From the LC analysis, it was found out that this product was a mixture of 1,6-diphenylpyrene, 1,8-diphenylpyrene, 1,3,6-triphenylpyrene and 1-phenylpyrene. Also, from the respective area values by LC, it was found out that it comprised 94.2% of diphenylpyrene mixture, 0.5% of monophenylpyrene, and 5.3% of triphenylpyrene.

The crude product was heated after adding ethanol, and then thermally filtered to separate a green filtrate and colorless crystals. About 2 g of the collected crystals were suspended in isopropyl alcohol while heating and refluxing, and then cooled to room temperature to collect crystals. With structural analysis by ¹H-NMR, it was confirmed that the collected crystals are 1,6-diphenylpyrene. In the LC analysis, 1,6-diphenylpyrene was one of their components, and the collected crystals had a purity of not less than 99.8%. The yielded amount was 0.92 g and the yield was 23.3%.

¹H-NMR (CDCl₃, 400 MHz)

1,6-diphenylpyrene . . . δ8.22 (d, 2H, J=7.60), 8.20 (d, 2H, J=9.20), 8.05 (d, 2H, J=9.20), 8.04 (d, 2H, J=8.40), 7.65-7.63 (m, 4H), 7.61-7.55 (m, 4H), 7.53-7.47 (m, 4H)

The filtrate obtained by thermally filtering with ethanol was condensed, and then washed by suspension in hexane to separate a white solid by filtration. The resulting filtrate was purified with GPC, and the fraction after removing triphenylpyrene mixed therein was condensed in an evaporator, and washed with methanol to yield a white solid. It was confirmed that the solid obtained from ¹H-NMR was 1,8-diphenylpyrene. In the LC analysis, 1,8-diphenylpyrene was one of its components, and its purity was 99.8%. The yielded amount was 0.32 g and the yield was 1%.

¹H-NMR (CDCl₃, 400 MHz)

1,8-diphenylpyrene . . . δ8.25 (d, 2H, J=7.60), 8.13 (s, 2H), 8.12 (s, 2H), 8.00 (d, 2H, J=7.60), 7.66-7.62 (m, 4H), 7.57-7.52 (m, 4H), 7.49-7.43 (m, 2H),

### Production Example 2

### Production of bis(trifluoromethylphenyl)pyrene (Compound 3)

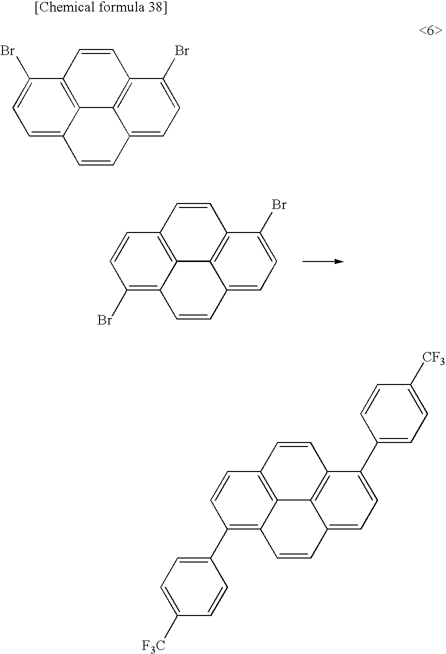

2.50 g of a 1:1 mixture of 1,6-dibromopyrene and 1,8-dibromopyrene, 3.98 g (3.0 equivalent weight with respect to dibromopyrenes) of p-trifluoromethylphenyl borate (reagent made by Wako Pure Chemical Industries, Ltd.), and 4.45 g of Na₂CO₃ (reagent made by Kanto Chemical Co., Inc.) were put into a 200 ml three-necked flask including a reflux condenser tube, a three-way cock connected to a nitrogen line, a thermometer and a rotor. 50 ml of toluene (reagent made by Junsei Chemical Co., Ltd.), 20 ml of ethanol (reagent made by Junsei Chemical Co., Ltd.), and 10 ml of desalted water were added and suspended. The operation of degassing the resulting suspension and returning the pressure to normal pressure with nitrogen was repeated five times. Then, the reaction mixture was bubbled with nitrogen for nitrogen purging in the system. 0.41 g of tetrakistriphenylphosphine palladium (reagent made by Tokyo Chemical Industry Co., Ltd.) was then added, and the system was stirred while heating at 80° C. for 10 hours in an oil bath, and then cooled to room temperature.

The precipitated reaction mixture was collected by suction filtration, and the solid was washed by suspension in ethanol. The crude product thus obtained was heated while suspending it in chloroform, and the undissolved substances were filtered out by gusseted filter paper. Crystals obtained by condensing the filtrate were washed with methanol and collected by suction filtration. It was confirmed that the crystals obtained from ¹H-NMR were 1,6-bis(4-trifluoromethylphenyl)pyrene. In the LC analysis, 1,6-bis(4-trifluoromethylphenyl)pyrene was one of their components, and their purity was 99.1%. The yielded amount was 1.59 g and the yield was 46.6%. The content of 1,6-isomer was 93.2%.

¹H-NMR (CDCl₃, 400 MHz)

1,6-(4-trifluoromethylphenyl)pyrene . . . δ8.26 (d, 2H, J=7.60), 8.14 (d, 2H, J=9.20), 8.09 (d, 2H, J=9.60), 7.99 (d, 2H, J=8.40), 7.85 (d, 4H, J=8.00), 7.77 (d, 4H, J=8.00)

### Production Example 3

### Production of bis(p-butylphenyl)pyrene (Compound 4)

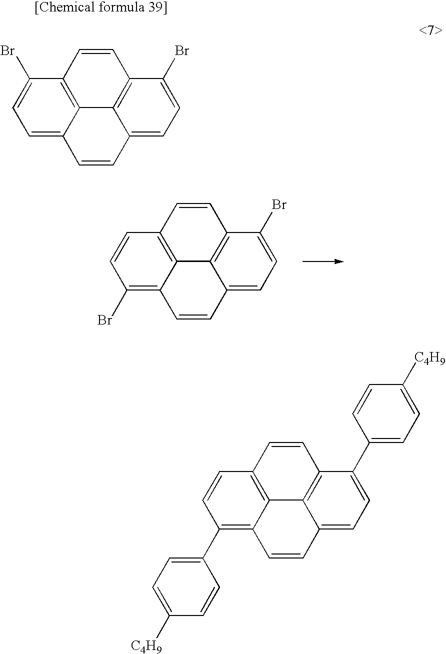

3.01 g of a 1:1 mixture of 1,6-dibromopyrene and 1,8-dibromopyrene, 4.5 g (3.0 equivalent weight with respect to dibromopyrenes) of p-butylphenyl borate (reagent made by Wako Pure Chemical Industries, Ltd.), and 5.30 g of Na₂CO₃ (reagent made by Kanto Chemical Co., Inc.) were put into a 200 ml three-necked flask including a reflux condenser tube, a three-way cock connected to a nitrogen line, a thermometer and a rotor. 50 ml of toluene (reagent made by Junsei Chemical Co., Ltd.), 20 ml of ethanol (reagent made by Junsei Chemical Co., Ltd.), and 10 ml of desalted water were added and suspended. The operation of degassing the resulting suspension and returning the pressure to normal pressure with nitrogen was repeated five times. Then, the reaction mixture was bubbled with nitrogen for nitrogen purging in the system. 0.50 g of tetrakistriphenylphosphine palladium (reagent made by Tokyo Chemical Industry Co., Ltd.) was then added, and the system was stirred while heating at 80° C. for 11 hours in an oil bath, and cooled to room temperature.

The reaction solution was separated into phases by adding 100 ml of chloroform and desalted water, and then the water phase was extracted with chloroform. The organic phases were combined with each other, dried over anhydrous magnesium sulfate, and condensed after removing a drying agent to yield a crude product. Inorganic substances were then removed from the crude product by column chromatography (SiO₂, chloroform) to yield a white solid.

The solid obtained was heated and washed by suspension in isopropyl alcohol, undissolved substances were filtered out by thermal filtration, and crystals precipitated from the filtrate were collected. They were combined and heated and washed by suspension in ethanol to yield white crystals. It was confirmed that the crystals obtained from ¹H-NMR were 1,6-bis(4-butylphenyl)pyrene. In the LC analysis, besides 1,6-bis(4-butylphenyl)pyrene, 1,8-bis(4-butylphenyl)pyrene was identified. The area ratio 1,6-compound: 1:8-compound was 98.0:2.0. The yielded amount was 0.99 g and the yield was 25.2%. The content of 1,6-isomer was 50.4%

¹H-NMR (CDCl₃, 400 MHz)

1,6-(4-butylphenyl)pyrene . . . δ8.22 (d, 2H, J=9.20), 8.19 (d, 2H, J=7.60), 8.04 (d, 2H, J=8.80), 7.99 (d, 2H, J=7.60), 7.56 (d, 4H, J=8.40), 7.39 (d, 4H, J=8.40), 2.78 (t, 4H, J=8.00), 1.77 (m, 4H), 1.51 (m, 4H), 1.02 (t, 6H, J=7.20)

### Production Example 4

### Production of bis(biphenyl)pyrene (Compound 5)

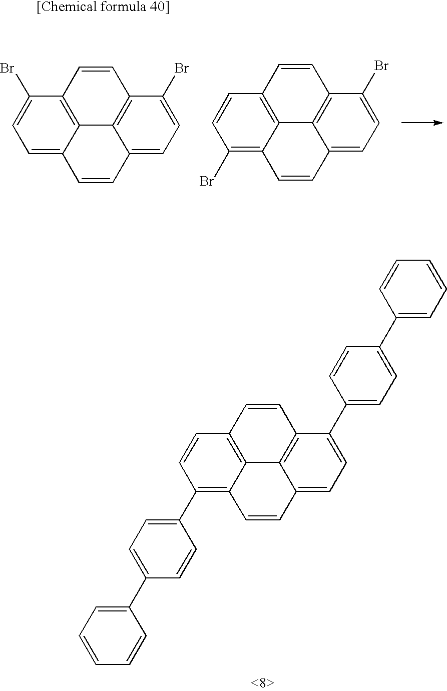

2.88 g of a 1:1 mixture of 1,6-dibromopyrene and 1,8-dibromopyrene, 4.75 g of 4-biphenyl borate (reagent made by Wako Pure Chemical Industries, Ltd.), and 5.10 g of Na₂CO₃ (reagent made by Kanto Chemical Co., Inc.) were put into a 200 ml three-necked flask including a reflux condenser tube, a three-way cock connected to a nitrogen line, a thermometer and a rotor. 110 ml of toluene (reagent made by Junsei Chemical Co., Ltd.), 20 ml of ethanol (reagent made by Junsei Chemical Co., Ltd.), and 10 ml of desalted water were added and suspended. The system of the reaction solution was purged with nitrogen. 0.46 g of tetrakistriphenylphosphine palladium (reagent made by Tokyo Chemical Industry Co., Ltd.) was then added, and the system was stirred while heating at 80° C. for 11 hours in an oil bath, and cooled to room temperature. The reaction solution was filtered, and the solid thus obtained was recrystallized twice with toluene to yield 0.86 g of yellow crystals.

¹H-NMR (CDCl₃, 400 MHz)

6-bis(4-4′-biphenyl)pyrene . . . δ8.28 (d, 2H, J=9.60), 8.23 (d, 2H, J=8.0), 8.08 (d, 2H, J=9.6), 8.04 (d, 2H, J=8.0), 7.81 (d, 4H, J=8.4), 7.76-7.73 (m, 8H), 7.54-7.50 (m, 4H), 7.34-7.36 (m, 2H)

### Production Example 5

### Production of bis(styryl)pyrene (Compound 6)

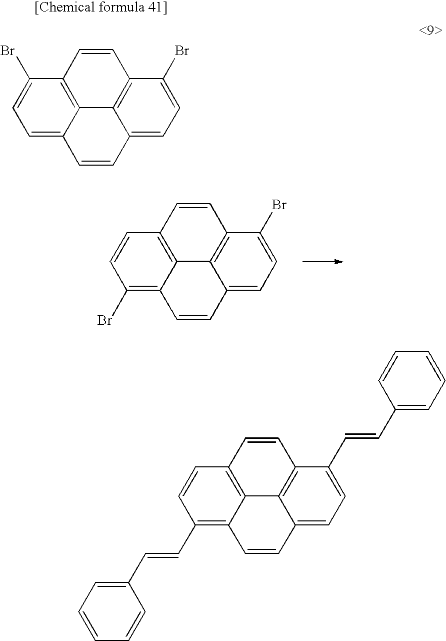

3.09 g of a 1:1 mixture of 1,6-dibromopyrene and 1,8-dibromopyrene, 4.88 g of styryl borate (made by Aldrich Co.), and 3.35 g of Na₂CO₃ (reagent made by Kanto Chemical Co., Inc.) were put into a 200 ml three-necked flask including a reflux condenser tube, a three-way cock connected to a nitrogen line, a thermometer and a rotor. 110 ml of toluene (reagent made by Junsei Chemical Co., Ltd.), 20 ml of ethanol (reagent made by Junsei Chemical Co., Ltd.), and 10 ml of desalted water were added and suspended. The system of the reaction solution was purged with nitrogen. 0.46 g of tetrakistriphenylphosphine palladium (reagent made by Tokyo Chemical Industry Co., Ltd.) was then added, and the system was stirred while heating at 80° C. for 11 hours in an oil bath, and cooled to room temperature. The reaction solution was filtered, and the solid thus obtained was recrystallized twice with toluene to yield 0.51 g of yellow crystals.

¹H-NMR (CDCl₃, 400 MHz)

6-bisstyrylpyrene . . . δ8.48 (d, 2H, J=8.80), 8.32 (d, 2H, J=8.0), 8.19 (d, 2H, J=15.6), 8.18 (d, 2H, J=8.0), 8.11 (d, 2H, J=9.6), 7.69 (d, 2H, J=8.4), 7.47-7.43 (m, 4H), 7.38-7.34 (m, 4H)

### Production Example 6

### Production of bis(2-naphthyl)pyrene (Compound 7)

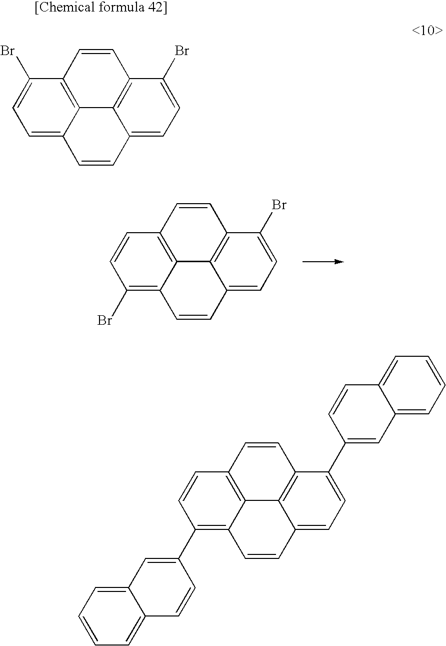

3.60 g of a 1:1 mixture of 1,6-dibromopyrene and 1,8-dibromopyrene, g of naphthyl borate (made by Aldrich Co.), and 6.36 g of Na₂CO₃ (reagent made by Kanto Chemical Co., Inc.) were put into a 200 ml three-necked flask including a reflux condenser tube, a three-way cock connected to a nitrogen line, a thermometer and a rotor. 120 ml of toluene (reagent made by Junsei Chemical Co., Ltd.), 25 ml of ethanol (reagent made by Junsei Chemical Co., Ltd.), and 14 ml of desalted water were added and suspended. The system of the reaction solution was purged with nitrogen. 0.58 g of tetrakistriphenylphosphine palladium (reagent made by Tokyo Chemical Industry Co., Ltd.) was then added, and the system was stirred while heating at 80° C. for 11 hours in an oil bath, and cooled to room temperature. The reaction solution was filtered, and the solid thus obtained was recrystallized with toluene to yield 1.5 g of yellow crystals.

¹H-NMR (CDCl₃, 400 MHz)

δ8.25 (d, 2H, J=8.80), 8.17 (d, 4H, J=3.2), 8.10 (d, 4H, J=8.80), 8.00 (d, 2H, J=8.4), 7.95-8.00 (m, 4H), 7.80 (dd, 2H, J=2.0, 8.4), 7.55-7.65 (m, 4H)

### Production Example 7

### Production of 1,6-bis(p-butylstyryl)pyrene (Compound 8)

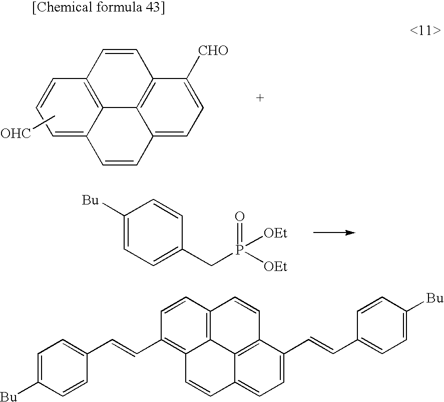

A 200 ml three-necked flask was fitted with a dropping funnel, a three-way cock connected to a nitrogen line, and a rotor, and the operation of drying under reduced pressure and nitrogen purging in the system was performed five times. 1.51 g of a 1:1 mixture of 1,6-diformylpyrene and 1,8-diformylpyrene and 4.14 g of p-butylbenezesulfonate were put into the reactor, and after performing nitrogen purging again, 100 ml of dried DMF (reagent made by Kanto Chemical Co., Inc.) was added and the mixture was stirred at room temperature. 4.4 ml of 5N—NaOMe/methanol solution (reagent made by Tokyo Chemical Industry Co., Ltd.) was dropwise added from the dropping funnel. After the end of the addition, the mixture was heated in an oil bath at the internal temperature of 80° C. for 10 hours.

After the reaction, precipitated needle crystals were collected by suction filtration, and washed by suspension in methanol to yield light yellow crystals. The thus collected crude crystals were recrystallized from toluene to yield light yellow crystals. The filtrate after recrystallization was condensed and separately recrystallized from toluene to yield light yellow crystals. From ¹H-NMR, it was confirmed that the crystals obtained in both operations were 1,6-bis(p-butylstyryl)pyrene. In the LC analysis, besides 1,6-bis(4-butylstyryl)pyrene, 1,8-bis(4-butylstyryl)pyrene was also identified. The area ratio 1,6-compound: 1:8-compound was 99.8:0.2. The yielded amount was 0.82 g and the yield was 27.0%. The content of 1,6-isomer was 54.0%

¹H-NMR (CDCl₃, 400 MHz)

1,6-bis(p-butylstyryl)pyrene . . . δ8.47 (d, 2H, J=8.80), 8.31 (d, 2H, J=7.60), 8.18-8.13 (m, 4H), 8.11 (d, 2H, J=9.20), 7.61 (d, 4H, J=8.00), 7.35 (d, 2H, J=15.60), 7.26 (d, 4H, J=8.00), 2.68 (t, 4H, J=7.60), 1.69-1.61 (m, 4H), 1.45-1.35 (m, 4H), 0.98 (t, 6H, J=7.20)

### Production Example 8

### Production of 1,6-bis(4-trifluoromethylstyryl)-pyrene (Compound 9)

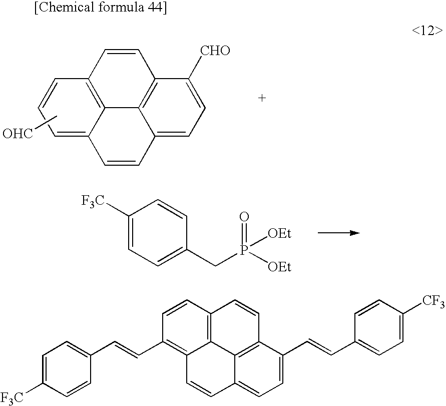

A 200 ml three-necked flask was fitted with a dropping funnel, a three-way cock connected to a nitrogen line, and a rotor, and the operation of drying under reduced pressure and nitrogen purging in the system was performed five time. 1.50 g of a 1:1 mixture of 1,6-diformylpyrene and 1,8-diformylpyrene and 3.80 g of p-trifluoromethylbenezensulfonate were put into the reactor, and after performing nitrogen purging again, 100 ml of dried DMF (reagent made by Kanto Chemical Co., Inc.) was added and the mixture was stirred at room temperature. 4.0 ml of 5N—NaOMe/methanol solution (reagent made by Tokyo Chemical Industry Co., Ltd.) was dropwise added from the dropping funnel. After stirring the mixture for 5 hours, the mixture was heated in an oil bath at the internal temperature of 80° C. for 3 hours.

After the reaction, precipitated needle crystals were collected by suction filtration, and washed by suspension in methanol to yield light yellow crystals. The thus collected crude crystals were washed by suspension in chloroform while heating, and undissolved solids were collected by suction filtration. The thus collected solids were recrystallized from toluene to yield white flaky crystals. From ¹H-NMR, it was confirmed that the crystals obtained were 1,6-bis(4-trifluoromethylstyryl)pyrene. In the LC analysis, it was confirmed that the crystals consisted of 1,6-bis(4-trifluoromethylstyryl)pyrene only. The yielded amount was 0.50 g and the yield was 16.1%. The content of 1,6-isomer was 32.2%

¹H-NMR (CDCl₃, 400 MHz)

1,6-bis(4-trifluoromethylstyryl)pyrene . . . δ8.51 (d, 2H, J=8.80), 8.36 (d, 2H, J=8.00), 8.32 (d, 2H, J=16.40), 8.23 (d, 2H, J=8.00), 8.17 (d, 2H, J=9.20), 7.80 (d, 4H, J=8.00), 7.70 (d, 4H, J=8.00), 7.40 (d, 2H, J=16.00)

### Production Example 9

### Production of 1,6-bis(4′-butylstilbene-4-yl)pyrene (Compound 10)

[Production of Raw Intermediate Material: 1,6-pyrenebis(pinacolate Diborane)]

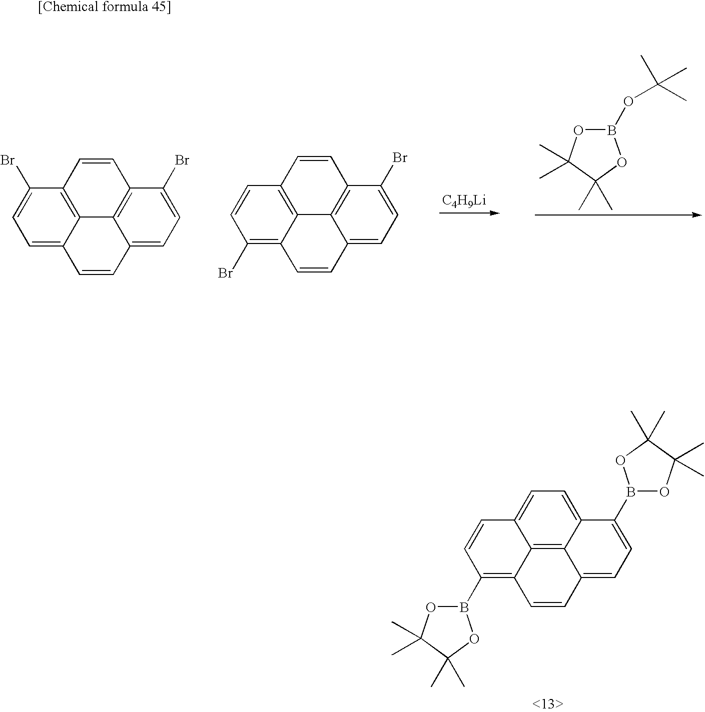

A 1000 ml four-necked flask was fitted with a dropping funnel, a three-way cock connected to a nitrogen line, and a low-temperature thermometer, a stirrer was mounted therein, and heat drying and nitrogen purging were performed five times using a heat gun under reduced pressure to produce a nitrogen atmosphere in the system. Then, 14.4 g of a 1:1 mixture of 1,6-dibromopyrene and 1,8-dibromopyrene and 300 ml of dried THF (reagent made by Kanto Chemical Co., Inc.) were added, and the reactor was cooled to −70° C. in a dry ice-acetone bath. 100 ml of n-butyllithium (1.06M, a reagent made by Kanto Chemical Co., Inc.) was dropwise added for 30 minutes. After the end of dropping, the system was further stirred for two hours while keeping the temperature at −70° C. 35 ml of Dioxaborolane (reagent made by Kanto Chemical Co., Inc.) was dropwise added from the dropping funnel for 15 minutes. During this process, heat build-up was confirmed and the internal temperature rose from −70° C. to −60° C. After the end of dropping of Dioxaborolane, the system was further stirred for 30 minutes while cooling. Then, the cooling bath was removed, and the system was gradually heated to room temperature and left to stand overnight. White crystals were precipitated in a light yellow solution. They were filtered by suction filtration. The crystals were stirred with 10 ml of 1N—HCl and 100 ml of water. The thus obtained crystals were collected by suction filtration to yield a wet cake. The cake obtained was washed by suspension in THF and dried under reduced pressure. It was confirmed that yellow crystals obtained from ¹H-NMR were 1,6-pyrenebis(pinacolate diborane). The yielded amount was 4.6 g and the yield was 25.0%. The content of 1,6 compounds was 60%.

¹H-NMR (CDCl₃, 400 MHz)

1,6-pyrenebis(pinacolate diborane) . . . δ9.1 (d, 2H, J=9.20), 8.52 (d, 2H, J=7.60), 8.18 (d, 2H, J=7.60), 8.19 (d, 2H, J=9.2), 1.49 (s, 12H)

[Production of Raw Intermediate Material: 4-bromobenzyl Diethyl Phosphonate]

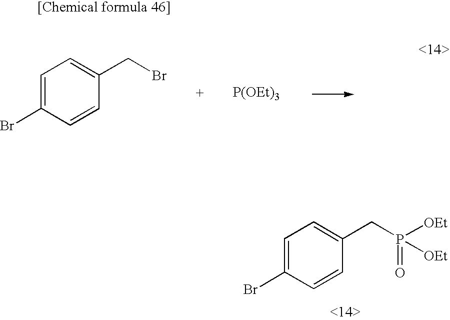

A three-way cock connected to a nitrogen line was connected to a 100 ml eggplant flask, and a rotor was put therein. 10.00 g of bromobenzyl bromide (reagent made by Aldrich Co.) and 20 ml of triethyl phosphite (reagent made by Wako Pure Chemical Industries, Ltd.) were put into the flask, and stirred while heating in an oil bath at 100° C. for four hours. It was confirmed that the raw materials disappeared from TLC and one high-polarity component was produced.

After the end of the reaction, any excess triethyl phosphite is distilled off to yield as a residue a light yellowish transparent oil substance. From ¹H-NMR, it was confirmed that this residue was a mixture of 4-bromobenzyl diethyl phosphonate and the remaining triethyl phosphite. The yielded amount was 15.3 g and the yield was about 80%.

¹H-NMR (CDCl₃, 300 MHz)

4-bromobenzyl diethyl phosphonate . . . δ7.44 (d, 2H, 7.81), 7.19 (d, 2H, J=8.41), 4.06 (m, 4H), 3.12 (d, 2H, J=21.62), 1.27 (t, 3H, J=7.20)

[Production of Raw Intermediate Material: 4-bromo-4′-butylstilbene]

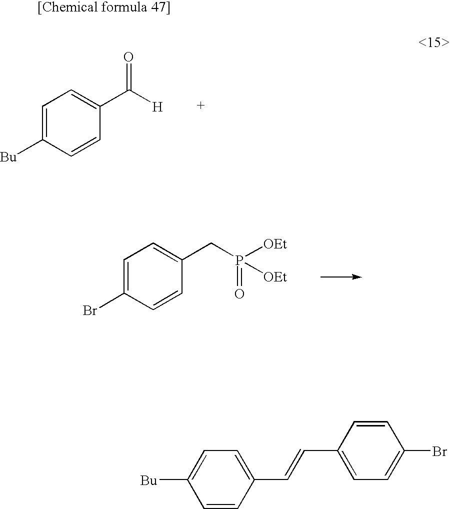

A rotor was put in a 100 ml three-necked flask, and a dropping funnel, a three-way cock connected to a nitrogen line and thermometer were mounted to the flask. 4.89 g of 4-butylbenzaldehyde (reagent made by Tokyo Chemical Industry Co., Ltd.) and 10.03 g of 4-bromobenzyl diethyl phosphonate were put into the flask, and after the interior of the reactor was purged with nitrogen, 50 ml of dried DMF (reagent made by Wako Pure Chemical Industries, Ltd.) was added and the mixture was stirred at room temperature. 6.5 ml of sodium methoxide/methanol solution (reagent made by Wako Pure Chemical Industries, Ltd.) was dropwise added slowly for two minutes from the dropping funnel. After the end of dropping, the mixture was stirred while heating in an oil bath at 40° C. for four hours.

After the end of the reaction, 200 ml of desalted water and 100 ml of toluene were added to the reaction solution to separate the solution into phases. The water phase was then extracted twice with 100 ml of toluene. The organic phases were combined and condensed. The resulting waxy solid was washed by suspension in hexane to yield white crystals. From ¹H-NMR, it was confirmed that the crystals were 4-bromo-4′-butylstilbene. The yielded amount was 6.17 g and the yield was 68.4%.

¹H-NMR (CDCl₃, 300 MHz)

4-bromo-4′-butylstilbene . . . δ7.47-7.34 (m, 6H), 7.18 (d, 2H, J=8.11), 7.10 (d, 1H, J=16.20), 7.00 (J=16.20), 2.63 (t, 2H, J=7.51), 1.65-1.55 (m, 2H), 1.40-1.31 (m, 2H), 0.95 (t, 3H, J=7.51)

[Production of 1,6-bis(4′-butylstilbene-4-yl)pyrene (Compound 10)]

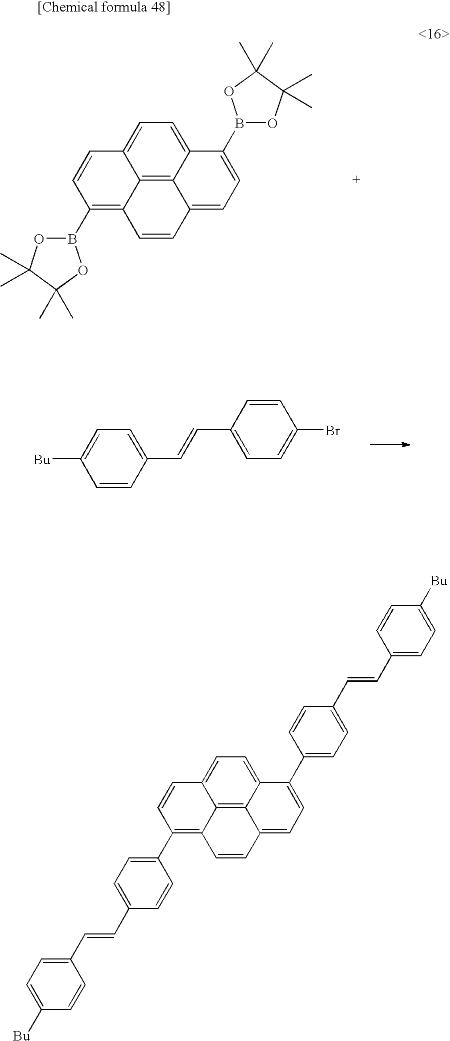

A rotor was put in a 200 ml three-necked flask, and a reflux condenser tube, a three-way cock connected to a nitrogen line, and a thermometer was mounted to the flask. 2.01 g of 1,6-pyrenebis(pinacolate diborane), 4.23 g of 4-bromo-4′-butylstilbene, 2.86 g of sodium carbonate (reagent made by Wako Pure Chemical Industries, Ltd.), 50 ml of toluene (reagent made by Wako Pure Chemical Industries, Ltd.), 20 ml of ethanol (reagent made by Wako Pure Chemical Industries, Ltd.), and 5 ml of desalted water were put into the flask. After performing degassing under reduced pressure and nitrogen purging five times, nitrogen was passed for 30 minutes through the mixture. After degassing, 0.30 g of tetrakistriphenylphosphine palladium (reagent made by Wako Pure Chemical Industries, Ltd.) was added, and the mixture was stirred while heating in an oil bath at 80° C. for 14 hours.

100 ml of desalted water and 100 ml of chloroform were added to the reaction solution to separate the solution into phases. The water phase was then extracted twice with 100 ml of chloroform. The solvent was distilled off, and the extract was dissolved into toluene at a hot reflux temperature, and thermally filtered. A yellow solid was collected by suction filtration from the filtrate obtained after removing undissolved inorganic substances. From ¹H-NMR, it was confirmed that the yellow crystals were 1,6-bis(4′-butylstilbene-4-yl)pyrene. The yielded amount was 2.06 g and the yield was 69.5%.

¹H-NMR (CDCl₃, 300 MHz)

1,6-bis(4′-butylstilbene-4-yl)pyrene . . . δ8.26 (d, 2H, J=9.31), 8.22 (d, 2H, J=8.11), 8.07 (d, 2H, J=9.30), 8.02 (d, 2H, J=7.81), 7.73 (d, 4H, J=8.41), 7.66 (d, 4H, J=8.41), 7.52 (d, 4H, J=8.11), 7.22-7.20 (m, 8H), 2.67 (t, 4H, J=7.51), 1.69-1.60 (m, 4H), 1.48-1.32 (m, 4H), 0.98 (t, 6H, J=7.20)

[Production of 2,7-bis(4-n-hexylphenyl)pyrene (Compound 11)]

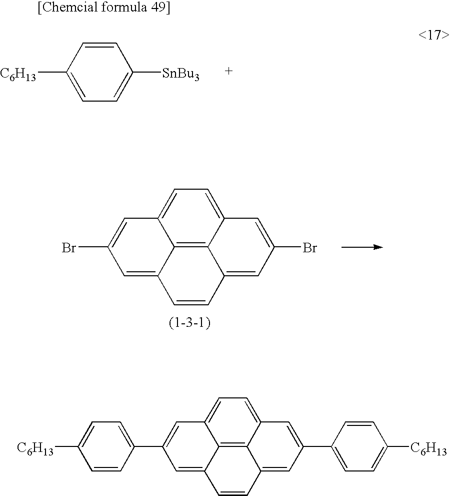

In an argon atmosphere, a toluene (35 ml) solution of 2,7-dibromopyrene (504 mg, 1.4 mmol), 1-(tri-n-butylstanyl)-4-n-hexylbenzene (1.90 g, 4.2 mmol), and tetrakis(triphenylphosphine) palladium (0) (97 mg, 0.084 mmol) was stirred while heating at 120° C. for 24 hours. The reaction solution was subjected to Celite filtration and condensed under reduced pressure. The resulting crystalline product was recrystallized from a toluene solution to yield an opaque white plate-crystalline captioned compound (483 mg, 0.92 mmol). The yield was 66%.

¹H-NMR (400 MHz, CDCl₃) . . . δ8.39 (s, 4H), 8.13 (s, 4H), 7.82 (d, J=8.0 Hz, 4H), 7.38 (d, J=8.0 Hz, 4H), 2.72 (t, J=7.8 Hz, 4H), 1.71 (m, 4H), 1.45-1.34 (m, 12H), 0.91 (t, J=7.0 Hz, 6H)

¹³C-NMR (100 MHz CDCl₃) . . . δ142.43, 138.93, 138.91, 131.58, 129.21, 127.97, 123.81, 35.82, 31.93, 31.68, 29.24, 22.81, 14.30

[Production of 2,7-bis[2-(6-n-hexyl)naphthyl]pyrene (Compound 12)]

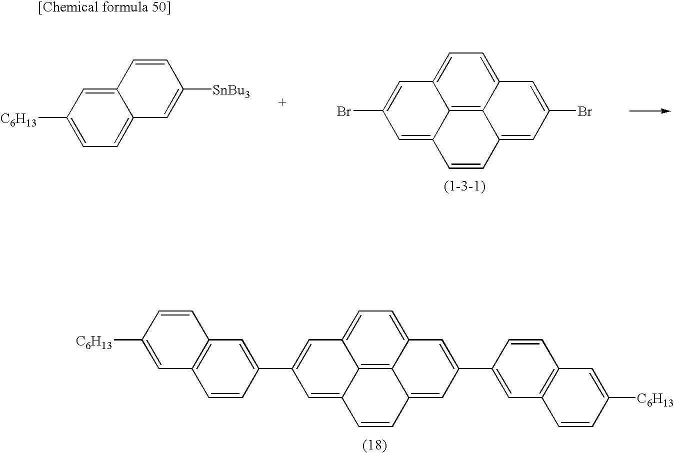

In an argon atmosphere, a toluene (35 ml) solution of 2,7-dibromopyrene (504 mg, 1.4 mmol), 2-(tri-n-butylstanyl)-6-n-hexylnaphthalene (1.92 g, 3.8 mmol), and tetrakis(triphenylphosphine) palladium (0) (97 mg, 0.084 mmol) was stirred while heating at 120° C. for 24 hours. The reaction solution was condensed under reduced pressure. After washing the residue with hexane, it was recrystallized from a toluene solution to yield the captioned compound in the form of white powder (315 mg, 0.51 mmol). The yield was 36%.

¹H-NMR (400 MHz, CDCl₃) . . . δ8.54 (s, 4H), 8.32 (br, 2H), 8.20 (s, 4H), 8.04 (dd, J=8.4/1.6 Hz, 2H), 7.97 (d, J=8.4 Hz, 2H), 7.92 (d, J=8.4 Hz, 2H), 7.70 (br, 2H), 7.42 (dd, J=8.4/1.2 Hz, 2H), 2.83 (t, J=7.6 Hz, 4H), 1.75 (m, 4H), 1.43-1.33 (m, 12H), 0.91 (t, J=7.0 Hz, 6H)

¹³C-NMR (100 MHz CDCl₃) . . . δ141.04, 139.13, 138.11, 133.05, 132.47, 131.75, 128.33, 128.22, 128.13, 126.72, 126.36, 126.27, 124.21, 36.36, 31.94, 31.52, 29.21, 22.80, 14.30

[Production of 2,7-bis(4-n-hexylphenylethynyl)pyrene (Compound 13)]

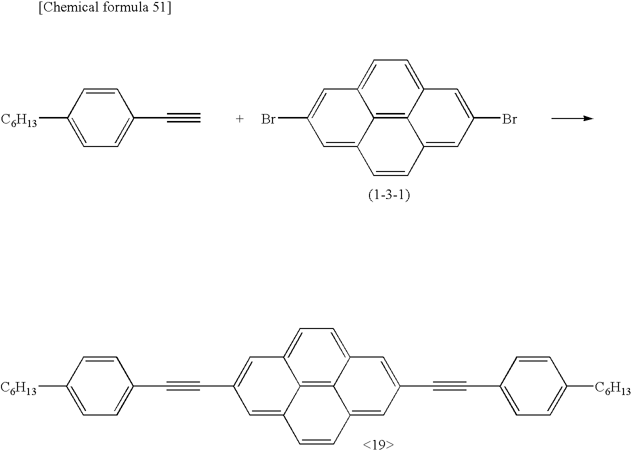

In an argon atmosphere, a toluene (35 ml) solution of 2,7-dibromopyrene (504 mg, 1.4 mmol), 1-ethynyl-4-n-hexylbenzene (634 mg, 3.4 mmol), tetrakis(triphenylphosphine) palladium (0) (97 mg, 0.084 mmol), copper iodide (8 mg, 0.042 mmol), and diisopropylamine (344 mg, 3.4 mmol) was stirred while heating at 120° C. for 24 hours. The reaction solution was poured into ice water, and after washing an extracted organic phase with saline water, it was dried with anhydrous magnesium sulfate. After condensing the residue under reduced pressure, diethyl ether was added thereto to crystallize it, thereby yielding the captioned compound in the form of yellow powder (335 mg, 0.59 mmol). The yield was 42%.

¹H-NMR (400 MHz, CDCl₃) . . . δ8.32 (s, 4H), 8.04 (s, 4H), 7.56 (d, J=8.0 Hz, 4H), 7.22 (d, J=8.0 Hz, 4H), 2.65 (t, J=7.8 Hz, 4H), 1.64 (m, 4H), 1.38-1.29 (m, 12H), 1. ee (t, J=7.8 Hz, 4H), 0.90 (t, J=7.0 Hz, 6H)

¹³C-NMR (100 MHz CDCl₃) . . . δ143.84, 131.80, 131.30, 128.72, 128.20, 127.74, 123.99, 121.48, 120.46, 90.57, 89.42, 36.12, 31.87, 31.39, 29.11, 22.76, 14.26

[Production of Raw Intermediate Material: 1,6-bis(3-methyl-3-hydroxy-1-ethynyl)pyrene]

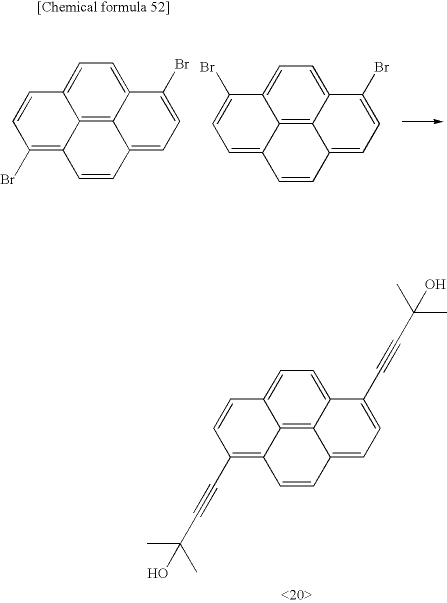

10.80 g of dibromopyrene of which the mixture ratio of 1,6-compound to 1,8-compound is 4:6, 0.21 g of copper iodate (reagent made by Wako Pure Chemical Industries, Ltd.; purity 95%), and 0.57 g of dichlorobis(triphenylphosphine)palladium (2) (reagent made by Wako Pure Chemical Industries, Ltd.) were put into a 500 ml three-necked flask including a reflux condenser tube, a three-way cock connected to a nitrogen line, and a septum cap. Then, the interior of the reactor was purged with nitrogen.

300 ml of diethylamine (reagent made by Wako Pure Chemical Industries, Ltd.; purity 99%) was added using a syringe, and nitrogen was passed through the solution for 20 minutes for degassing. After degassing, 7.5 ml of 2-methyl-3-butyne-2-ol (reagent made by Wako Pure Chemical Industries, Ltd.; purity 98%) was added, and the mixture was stirred while heating in an oil bath at 55° C. for 10 hours.

After the end of the reaction, the solvent was distilled off under reduced pressure with a rotary evaporator. The residue was washed with chloroform (reagent made by Wako Pure Chemical Industries, Ltd.) to yield a white solid. From ¹H-NMR, it was confirmed that this white solid was 1,6-bis(3-methyl-3-hydroxy-1-butynyl)pyrene. The yielded amount was 4.2 g. The yield was 96% relative to the raw material 1,6-dibromopyrene.

¹H-NMR (CDCl₃, 300 MHz,) . . . δ8.52 (d, 2H, J=9.1), 8.12 (m, 6H), 2.20 (s, 2H), 1.81 (s, 12H)

[Production of Raw Intermediate Material: 1,6-diethynylpyrene]

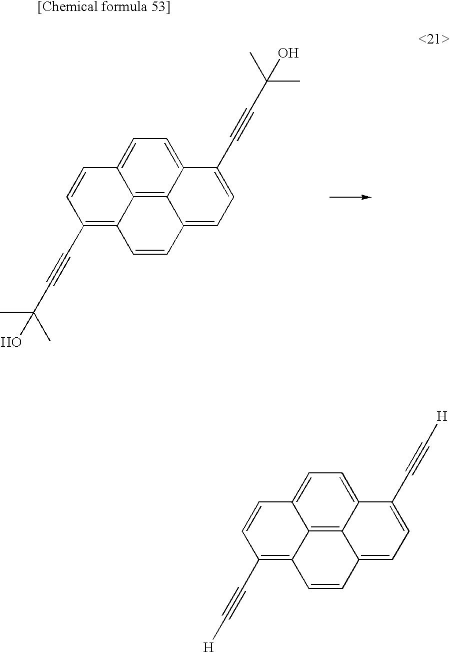

4.2 g of 1,6-bis(3-methyl-3-hydroxy-1-butynyl)pyrene, 4.2 g of sodium hydroxide (reagent made by Wako Pure Chemical Industries, Ltd.; purity 96%), and 170 ml of toluene (reagent made by Wako Pure Chemical Industries, Ltd.) were put into a 300 ml eggplant flask which is equipped with a reflux condenser tube and in which a rotor is mounted. The mixture was stirred while heating in an oil bath at 105° C. for 70 hours.

After the end of the reaction, undissolved solids were removed by thermal filtration. The filtrate thus obtained was condensed under reduced pressure in a rotary evaporator to yield red solids. The solids thus obtained were recrystallized from toluene to obtain red needle crystals. From ¹H-NMR, it was confirmed that the red needle crystals were 1,6-diethynylpyrene. The yielded amount was 1.79 g. The yield was 60%.

¹H-NMR (CDCl₃, 300 MHz,) . . . δ8.62 (s, 2H), 8.13-8.22 (m, 6H), 3.64 (s, 2H)

[Production of Raw Intermediate Material: 1,6-pyrenebis(vinyl Borate Catechol Ester)

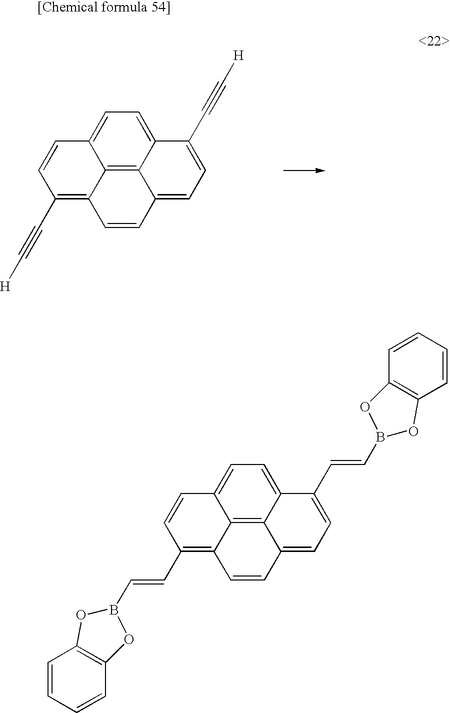

2.10 g of 1,6-diethynylpyrene was put into a 100 ml two-necked flask which is equipped with a reflux condenser tube and a three-way cock connected to a nitrogen line, and in which a rotor is mounted. The reactor was then purged with nitrogen. 60 ml of anhydrous toluene (reagent made by Wako Pure Chemical Industries, Ltd.), and 25 ml of a THF solution of catecholborane (reagent made by Aldrich Co.; concentration 1 M) were added with a syringe. The mixture was then stirred while heating in an oil bath at 110° C. for 40 hours.

Solids produced by the reaction were collected by suction filtration, and washed with toluene (reagent made by Wako Pure Chemical Industries, Ltd.) to yield yellow solids. Due to low solubility in chloroform, ¹H-NMR of this compound was not measurable. The yielded amount was 2.60 g. The yield was 64%.

[Production of 1,6-bis(4-methylstyryl)pyrene (Compound 14)]

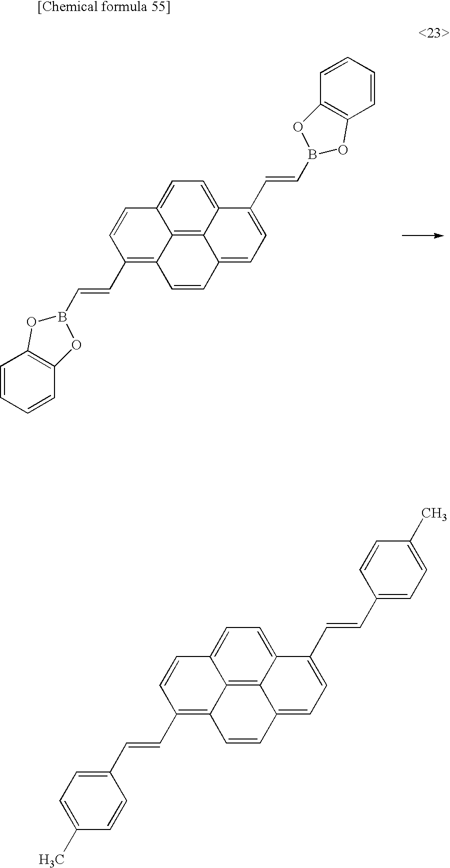

1.30 g of 1,6-pyrenebis((E)-vinyl borate catechol ester), 0.15 g of tetrakis(triphenylphosphine)palladium (0) (reagent made by Wako Pure Chemical Industries, Ltd.), and 1.70 g of sodium carbonate (reagent made by Wako Pure Chemical Industries, Ltd.; purity 99.5%) were put into a 200 ml two-necked flask which is equipped with a reflux condenser tube and a three-way cock connected to a nitrogen line, and in which a rotor is mounted. The system was then purged with nitrogen. 60 ml of toluene (reagent made by Wako Pure Chemical Industries, Ltd.), 24 ml of ethanol (reagent made by Wako Pure Chemical Industries, Ltd.), and 6 ml of desalted water were put into this reactor, and nitrogen was passed through the solution for 30 minutes for degassing. 1.0 ml of p-bromotoluene (reagent made by Wako Pure Chemical Industries, Ltd.; purity 95%) was added using a syringe, and the mixture was stirred while heating in an oil bath at 90° C. for 12 hours.

After the end of the reaction, the solvent was distilled off under reduced pressure with a rotary evaporator. Then, the residue was dissolved into hot toluene and passed through celite (reagent made by Wako Pure Chemical Industries, Ltd.) and Florisil (reagent made by Wako Pure Chemical Industries, Ltd.) to remove inorganic and undissolved components. The filtrate was condensed and recrystallized from o-dichlorobenzene (reagent made by Wako Pure Chemical Industries, Ltd.) to yield red crystals. From ¹H-NMR, it was confirmed that the red crystals were 1,6-bis(4-methylstyryl)pyrene. The yielded amount was 0.66 g. The yield was 57%.

¹H-NMR (CDCl₃, 270 MHz) . . . δ8.48 (2H, d, J=9.2), 8.31 (2H, d, J=7.8), 8.17 (d, 4H, J=8.10), 8.15 (2H, d, J=15.7), 8.11 (2H, d, J=9.5), 7.59 (2H, d, J=8.1), 7.33 (2H, d, J=8.1), 7.25 (4H, d), 2.41 (6H, s)

[Production of 1,6-bis(ethylstyryl)pyrene (Compound 15)

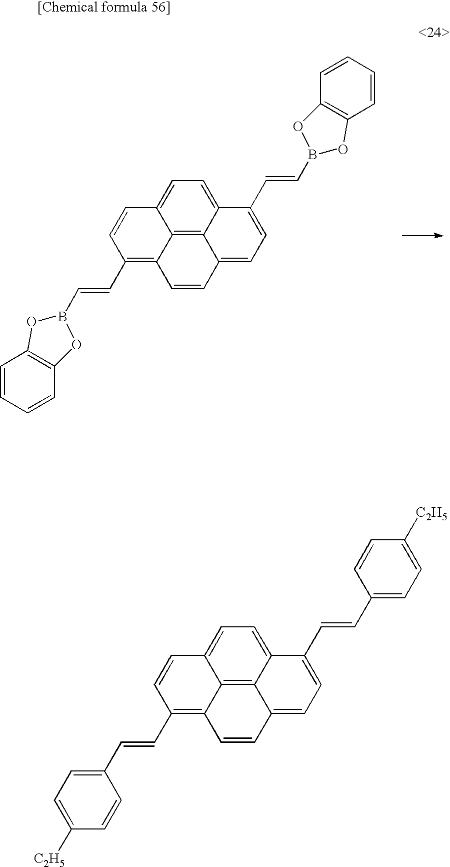

2.00 g of 1,6-pyrenebis((E)-vinyl borate catechol ester), 0.24 g of tetrakis(triphenylphosphine)palladium (0) (reagent made by Wako Pure Chemical Industries, Ltd.), and 2.60 g of sodium carbonate (reagent made by Wako Pure Chemical Industries, Ltd.; purity 99.5%) were put into a 200 ml two-necked flask which is equipped with a reflux condenser tube and a three-way cock connected to a nitrogen line, and in which a rotor is mounted. The system was then purged with nitrogen. 100 ml of toluene (reagent made by Wako Pure Chemical Industries, Ltd.), 40 ml of ethanol (reagent made by Wako Pure Chemical Industries, Ltd.), and 10 ml of desalted water were put into this reactor, and nitrogen was passed through the solution for 30 minutes for degassing. 1.7 ml of p-bromoethylbenzene (reagent made by Wako Pure Chemical Industries, Ltd.) was added using a syringe, and the mixture was stirred while heating in an oil bath at 90° C. for six hours.

After the end of the reaction, the solvent was distilled off under reduced pressure with a rotary evaporator. Then, the residue was dissolved into hot toluene and passed through celite (reagent made by Wako Pure Chemical Industries, Ltd.) and Florisil (reagent made by Wako Pure Chemical Industries, Ltd.) to remove inorganic and undissolved components. The filtrate was condensed and recrystallized from toluene (reagent made by Wako Pure Chemical Industries, Ltd.) to yield yellow crystals. From ¹H-NMR, it was confirmed that the yellow crystals were 1,6-bis(4-ethyl styryl)pyrene. The yielded amount was 1.12 g. The yield was 58%.

¹H-NMR (CDCl₃, 400 MHz) . . . δ8.47 (2H, d, J=9.6), 8.31 (2H, d, J=8.4), 8.17 (d, 2H, J=8.0), 8.16 (d, 2H, J=16.8), 8.10 (2H, d, J=9.2), 7.62 (2H, d, J=8.0), 7.34 (2H, d, J=16.0), 7.26 (4H, d), 2.70 (4H, m), 1.29 (t, 6H, J=8.0)

[Production of 1,6-bis(4-propyl styryl)pyrene (Compound 16)]

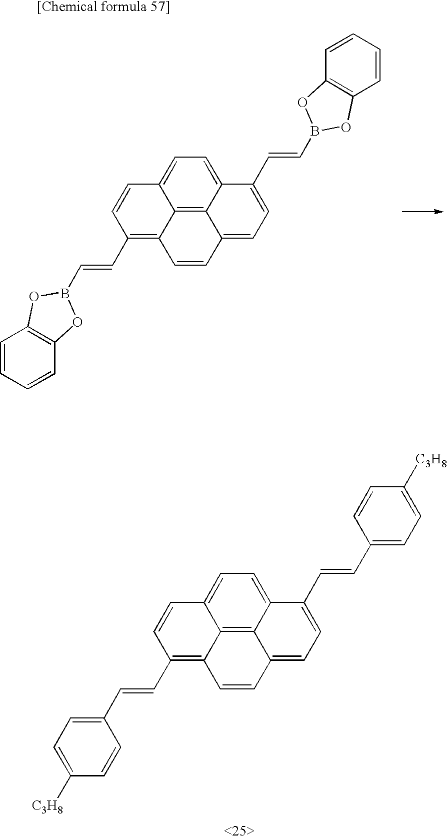

2.00 g of 1,6-pyrenebis((E)-vinyl borate catechol ester), 0.24 g of tetrakis(triphenylphosphine)palladium (0) (reagent made by Wako Pure Chemical Industries, Ltd.), and 2.60 g of sodium carbonate (reagent made by Wako Pure Chemical Industries, Ltd.; purity 99.5%) were put into a 200 ml two-necked flask which is equipped with a reflux condenser tube and a three-way cock connected to a nitrogen line, and in which a rotor is mounted. The system was then purged with nitrogen. 100 ml of toluene (reagent made by Wako Pure Chemical Industries, Ltd.), 40 ml of ethanol (reagent made by Wako Pure Chemical Industries, Ltd.), and 10 ml of desalted water were put into this reactor, and nitrogen was passed through the solution for 30 minutes for degassing. 1.9 ml of p-bromopropylbenzene (reagent made by Wako Pure Chemical Industries, Ltd.) was added using a syringe, and the mixture was stirred while heating in an oil bath at 90° C. for six hours.

After the end of the reaction, the solvent was distilled off under reduced pressure with a rotary evaporator. Then, the residue was dissolved into hot toluene and passed through celite (reagent made by Wako Pure Chemical Industries, Ltd.) and Florisil (reagent made by Wako Pure Chemical Industries, Ltd.) to remove inorganic and undissolved components. The filtrate was condensed and recrystallized from toluene (reagent made by Wako Pure Chemical Industries, Ltd.) to yield yellow crystals. From ¹H-NMR, it was confirmed that the yellow crystals were 1,6-bis(4-propyl styryl)pyrene. The yielded amount was 1.27 g. The yield was 63%.

¹H-NMR (CDCl₃, 400 MHz) . . . δ8.46 (2H, d, J=8.8), 8.30 (2H, d, J=7.6), 8.15 (d, 2H, J=8.8), 8.14 (d, 2H, J=15.2), 8.09 (2H, d, J=9.2), 7.60 (2H, d, J=8.4), 7.33 (2H, d, J=16.4), 7.25 (4H, d, J=8.0), 2.64 (4H, t, J=7.59), 1.69 (m, 4H, J=7.6), 0.99 (6H, t, J=7.20)

(Measurement/Calculation of Carrier Mobility, on/Off Ratio, EL Luminous Efficiency, and PL Luminous Efficiency)

The carrier mobility, the EL luminous efficiency, and the PL luminous efficiency were each measured and calculated as follows:

[Carrier mobility μ(cm²/Vₛ)]

A relational expression between the drain voltage (V_{d}) and the drain current of an organic semiconductor is represented by the following expression (1), and it increases linearly (linear area),

$$\begin{matrix}[ Expression 1 ] & \\ {I}_{d} = \frac{W}{L} μ {C}_{i} [ ( {V}_{g} - {V}_{T} ) {V}_{d} - \frac{1}{2} {V}_{d}^{2} ] & ( 1 )\end{matrix}$$

As V_{d} increases, I_{d} is saturated by the pinch-off of the channel, so that I_{d} becomes a constant value (saturated area) and is represented by the following expression (2):

$$\begin{matrix}[ Expression 2 ] & \\ {I}_{d} = \frac{W}{2 L} {μ}_{s a t} {{C}_{i} ( {V}_{g} - {V}_{T} )}^{2} & ( 2 )\end{matrix}$$

In the expressions (1) and (2), each of the symbols is as follows:

L: channel length [cm],

W: channel width [cm],

Cᵢ: electrostatic capacity [F/cm²] of the gate insulating film per unit area,

μₛₐₜ: mobility [cm²/Vs] in the saturate area,

I_{d}: drain current [A],

V_{d}: drain voltage [V],

V_{g}: gate voltage [V], and

V_{T}: gate threshold voltage [V] (which represents the following point: in a graph obtained by plotting the ½ power of the drain current (V_{dsat}_{^{1/2}}) versus the gate voltage (V_{g}) under a condition that the drain voltage (V_{d}) in the saturated area is constant, a point at which the asymptotic line therein intersects the transverse axis)

From the relationship between I_{d}_{^{1/2}} and V_{g} in this saturated area, the mobility (μ) in the organic semiconductor can be obtained.

In the present invention, under conditions that the pressure is set to the degree of vacuum of 5×10⁻³ and the temperature is set to room temperature, using a Semiconductor Parameter Analyzer (HP4155C, Agilent), the drain voltage was operated from 10 V to −100 V at increments of −1 V, and the gate voltage was operated from 0 V to −100 V at increments of −20 V to calculate the mobility using the expression (2).

(On/Off Ratio)

This ratio is the ratio of the maximum value of Id (Ion) when the gate voltage is −100 V to the minimum value of Id (loff) when the gate voltage is 0 V in the measurement of the carrier mobility.

(Vₜₕ and El Luminous Efficiency)

Vₜₕ is the voltage at the beginning of EL emission. For Vₜₕ and the EL luminous efficiency ηₑₓₜ, the above-mentioned transistor devices were used, and operations were made to set the drain voltage from 10 V to −100 V at increments of −1 V, and set the gate voltage from 0 to −100 V at increments of −20 V, and light emitted from the devices was measured with a photon counter (4155C, Semiconductor Parameter Analyzer, manufactured by Newport Co.) to calculate the voltage (Vₜₕ) at the beginning of light emission. The following expression (3) was used to convert the number of photons [CPS] obtained therein to the light fluxes [lw], and subsequently the following expression (4) was used to calculate the EL luminous efficiency ηₑₓₜ.

$$\begin{matrix}[ Expression 3 ] & \\ {X}_{P C} [ h v ] = \frac{5.71 \times {10}^{- 11} ( {N}_{P C} [ C P S ] - base ) \frac{4}{3} π {r}^{3} / \frac{h}{3} π {r}^{2}}{1.04 \times {10}^{6}} & ( 3 )\end{matrix}$$

[Expression 4]

ηₑₓₜ=(100×1239.7/λ×N_{PC}×X_{PC})/I_{d} (4)

In the expressions (3) and (4), each of the symbols is as follows:

N_{PC}: number of photons [CPS] measured with the photon counter (PC),

X_{PC}: numerical value obtained by converting the number of the photons to light fluxes [lw],

r: diameter [cm] of the cone or circle, and

h: distance [cm] between the photon counter and the sample.

(PL Peak and PL Luminous Efficiency)

The PL peak and PL luminous efficiency were calculated by depositing each of the transistor material according to the present invention on a quartz substrate to a thickness of 100 nm in the atmosphere of nitrogen to form a monolayer film, using an integrating sphere (IS-060, Labsphere Co.) to radiate a He—Cd laser (IK5651R-G, Kimmon Electric Co.) having a wavelength of 325 nm as an exciting ray, and then measuring a light-emitting Multi-channel photodiode (PMA-11, Hamamatsu Photonics Co.) from the samples.

(Measurement of HOMO and LUMO Energy Levels)

After forming a monolayer film on a quartz substrate by deposition to the thickness of 100 nm, the HOMO energy was calculated based on measurement using AC-1 (made by Riken Keiki Co., Ltd.). After measuring the band gap using a Hitachi spectrophotometer (UV-3500 made by Hitachi, Ltd.), the LUMO energy was calculated by adding the HOMO energy.

### Examples 1-15 and Comparative Example 1

Light-emitting transistor devices each illustrated in FIGS. 23 and 24 were produced under the following conditions:

Insulating film 5: A silicon oxide film 300 nm in thickness was formed by vapor deposition on a silicon substrate as an insulating film.

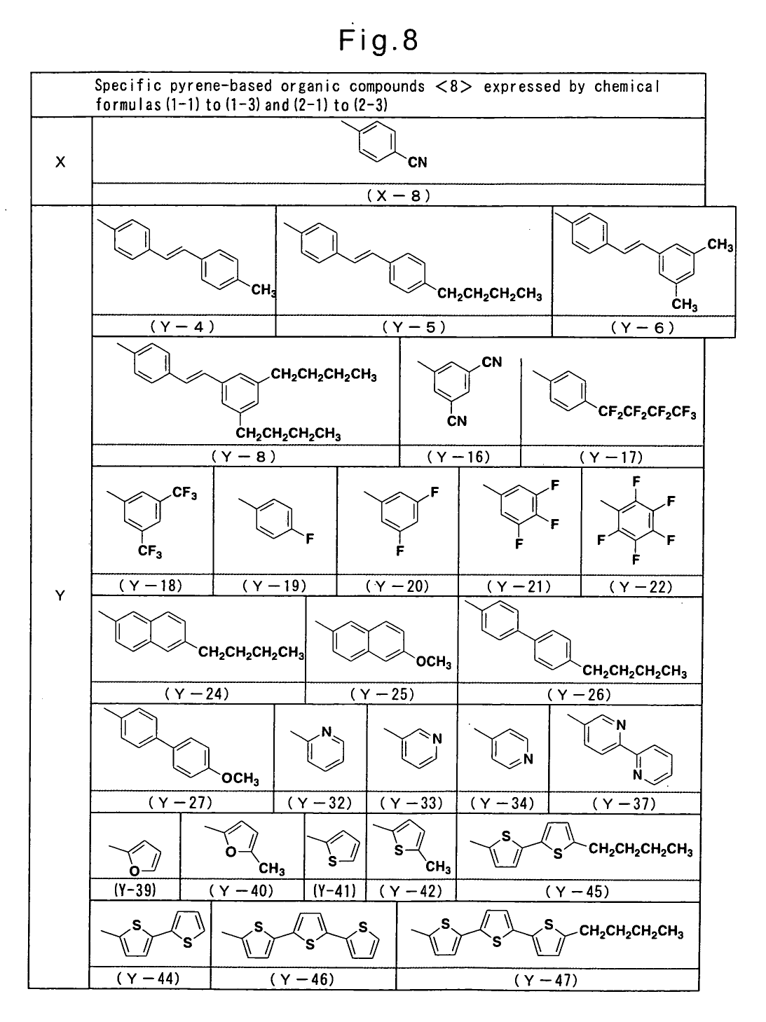

Source electrode 2 and Drain electrode 3: Electrodes (Au, thickness: 40 nm) each having a comb tooth shaped region made of twenty comb teeth were formed, and then the electrodes were arranged on an insulating film 5 to arrange the comb tooth shaped regions alternately, as illustrated in FIG. 8. At this time, a layer (1 nm) made of chromium was formed between the insulating film 5 and the two electrodes. About the channel regions (between the comb tooth shaped regions) at this time, the width was set to 25 μm and the length was set to 4 mm.

Light-emitting layer 1: A light-emitting layer 1 was formed by evaporating a transistor material selected from the compounds 1, 2, 4, 5, 6, 7, 8 and 11 produced in the above respective production examples to cover the surroundings of the insulating film, the source electrode 2 and the drain electrode 3.

For the resulting devices, the ratio of HOMO and LUMO levels, EL luminous efficiency, carrier mobility, PL peak, and Vₜₕ were measured by the above methods. Using the transistor devices comprising the respective compounds, the PL luminous efficiency was measured by the above method. The results are shown in Table 1.

“HMDS treatment” in Table 1 refers to a treatment by the below operation A.

Operation A: After forming the source electrode 2 and the drain electrode 3, the substrate was treated with UV ozone, HMDS as a surface treating agent was applied to the substrate, and the substrate was left to stand for two minutes. Then, the residue of HMDS was removed by air and a compound for use as the light emitting layer was deposited under vacuum.

### Comparative Example 1

Except that a comparative compound 1 shown by the below formula (6) was used as the compound for the light-emitting layer, a transistor device was produced in the same manner as in Example 1. For the resulting device, the ratio of HOMO and LUMO levels, EL luminous efficiency, carrier mobility, PL peak, and Vₜₕ were measured by the above methods. Using the transistor device comprising the comparative compound 1, the PL luminous efficiency was measured by the above method. The results are shown in Table 1.

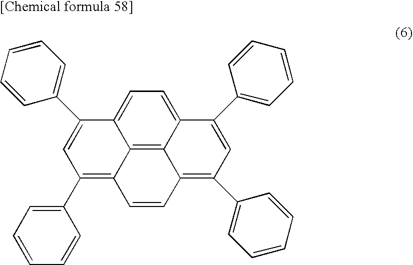

TABLE 1

| | | | | | | | PL | EL | |
|---|---|---|---|---|---|---|---|---|---|
| | | | | Carrier | | HOMO/LUMO | luminous | luminous | |
| | | | HMDS | mobility | PL peak | energy level | efficiency | efficiency | Vth |
| | | Compound | treatment | (cm²/V · s) | (nm) | (eV) | (%) | (%) | (V) |
| Example | 1 | Compound 1 | No | 8.5 × 10⁻⁴ | 447 | 5.4/2.3 | 81 | 1.0 × 10⁻² | −32 |
| | 2 | Compound 2 | No | 9.2 × 10⁻⁷ | 470 | 5.6/2.5 | 84 | 5.0 × 10⁻² | −17 |
| | 3 | Compound 4 | No | 2.5 × 10⁻⁸ | 419 | 6.0/3.0 | 67 | — | — |
| | 4 | Compound 5 | Yes | 1.2 × 10⁻⁵ | 465 | 5.6/2.8 | 80 | 1.0 × 10⁻¹ | −59 |
| | 5 | Compound 6 | No | 3.8 × 10⁻⁴ | 530 | 5.2/2.8 | 18 | 2.8 × 10⁻³ | −29 |
| | 6 | | Yes | 5.2 × 10⁻⁴ | — | — | — | 5.8 × 10⁻⁴ | — |
| | 7 | Compound 7 | No | 1.7 × 10⁻⁴ | 455 | 5.5/2.6 | 80 +/− 3 | — | −58 |
| | 8 | Compound 8 | No | 2.0 × 10⁻² | 520 | 5.0/2.4 | 5 | 3.0 × 10⁻³ | −37 |
| | 9 | | Yes | 6.7 × 10⁻² | — | — | — | 3.0 × 10⁻³ | — |
| | 10 | Compound 10 | No | 4.0 × 10⁻² | 521 | 5.0/2.4 | 5 | 3.2 × 10⁻⁵ | −29 |
| | 11 | Compound 11 | No | 1.0 × 10⁻³ | 418 | — | 16 | — | −82 |
| | 12 | | Yes | 2.0 × 10⁻² | 418 | — | 16 | 6.8 × 10⁻⁵ | −65 |
| | 13 | Compound 14 | No | 10. × 10⁻¹ | 520 | — | 8 | 5.7 × 10⁻⁴ | −23 |
| | 14 | Compound 15 | No | 4.0 × 10⁻² | 519 | — | 5 | 5.2 × 10⁻⁴ | −15 |
| | 15 | Compound 16 | No | 4.0 × 10⁻³ | 545 | — | 10 | 8.9 × 10⁻⁵ | −12 |
| Comparative | 1 | Comparative | No | 1.7 × 10⁻⁵ | — | 5.7/2.7 | 68 +/− 3 | <0.05 | — |
| Example | | compound 1 | | | | | | | |

### (Results)

From the above results, it was discovered that devices made of the compounds used in Examples were high in carrier mobility. While the compounds of Examples 1 and 4 were substantially equal in carrier mobility to the compound of Comparative Example 1, their PL luminous efficiency was extremely high.

From the comparison of examples which were subjected to and not subjected to HMDS treatment, it was discovered that the HMDS treatment improves carrier mobility.

When comparing Examples 10 and 13-15, among alkyl groups, methyl groups improve carrier mobility as well as the PL luminous efficiency and EL luminous efficiency. By selecting methyl groups as alkyl groups, purification by sublimation is easier and also, the purity can be improved.

### Examples 16-19 and Comparative Example 2

Transistor devices were produced in the same manner as in Example except that the respective transistor materials selected from the compounds 10 and 14-16 produced in the respective production examples were doped with 1 wt % of periflanthene produced according to the method described in J. Am. Chem. Soc., (1952), 74, 1075 and J. Am. Chem. Soc., (1996), 118, 2374 (compound shown by the below formula (7)), and their evaluation was made. The results are shown in Table 2.

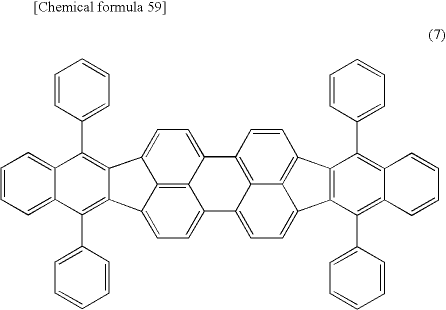

TABLE 2

| | | | | | | | | PL | EL | |
|---|---|---|---|---|---|---|---|---|---|---|
| | | | Periflanthene | | Carrier | | | luminous | luminous | |
| | | | added | HMDS | mobility | PL peak | On/Off | efficiency | efficiency | Vth |
| | | Compound | (1 wt %) | treatment | (cm²/V · s) | (nm) | ratio | (%) | (%) | (V) |
| Example | 16 | Compound 10 | Yes | No | 2.0 × 10⁻² | 615 | 10⁵ | 50 | 8.0 × 10⁻⁴ | −15 |
| | 17 | Compound 14 | Yes | No | 4.0 × 10⁻² | 615 | 10⁸ | 33 | 6.5 × 10⁻⁴ | −21 |
| | 18 | Compound 15 | Yes | No | 2.0 × 10⁻² | 615 | 10⁷ | 33 | 3.0 × 10⁻⁴ | −20 |
| | 19 | Compound 16 | Yes | No | 1.0 × 10⁻² | 615 | 10⁸ | 55 | 5.4 × 10⁻⁴ | −16 |
| Comparative | 2 | Comparative | Yes | No | 1.1 × 10⁻⁴ | 615 | 10³ | 78 | 2.1 × 10⁻³ | −30 |
| Example | | compound 1 | | | | | | | | |

### (Results)

Each of Examples 16-19 showed carrier mobility which is about 100 times or more than 100 times higher than in Comparative Example 1. This made it possible to significantly reduce the driving voltage. Further, the luminous efficiency was at least 10 times higher than the luminous efficiency attained with a single compound. Thus, the excitation energy of the compounds according to the present invention is efficiently transferred to guest materials. This indicates that the compounds according to the present invention can be used as host materials in the light-emitting layer.

1. A transistor material comprising a compound expressed by one of the below chemical formulas (1) and (2), 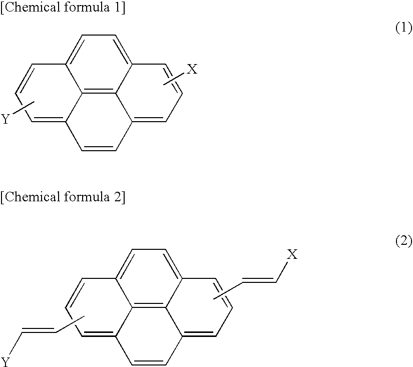
(In the formulas (1) and (2), each of X and Y is a substituent independent of the other and selected from an aromatic hydrocarbon group which may have a substituent, an aromatic heterocyclic group which may have a substituent, an alkyl group which may have a substituent, a cycloalkyl group which may have a substituent, an alkenyl group which may have a substituent, and an alkynyl group which may have a substituent, the substituents which X and Y may have are groups selected from alkyl groups having a carbon number of 1-20, alkoxy groups having a carbon number of 1-20, amino groups, boryl groups, silyl groups, cyano groups, aromatic hydrocarbon groups having a carbon number of 6-36, aromatic heterocyclic groups having a carbon number of 4-36 and halogen atoms, the compound expressed by either of the formulas (1) and (2) includes one pyrene ring in each molecule.)

2. The transistor material of claim 1 wherein said formula (1) is expressed by one of the below formulas (1-1) to (1-3), 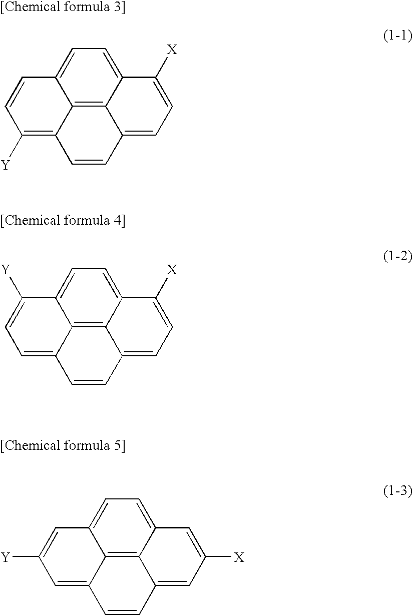
(X and Y in the formulas (1-1) to (1-3) are identical to X and Y in the formula (1), respectively, and the compound expressed by each of the formulas (1-1) to (1-3) includes one pyrene ring in each molecule.)

3. The transistor material of claim 1 wherein said formula (2) is expressed by one of the below formulas (2-1) to (2-3), 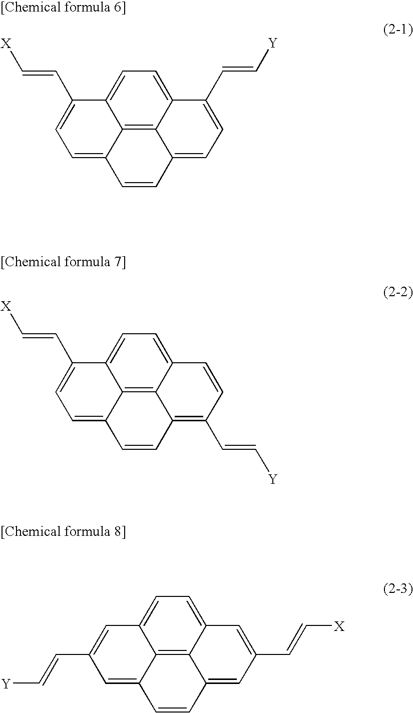
(X and Y in the formulas (2-1) to (2-3) are identical to X and Y in the formula (2), respectively, and the compound expressed by each of the formulas (2-1) to (2-3) includes one pyrene ring in each molecule.)

4. A light-emitting transistor material made of the transistor material of any of claims 1 to 3.

5. A light-emitting transistor device comprising a light-emitting layer comprising as a major component the light-emitting transistor material of claim 4 and capable of transporting holes and electrons as carriers, said light-emitting layer emitting light by recombination of the holes and the electrons; a hole injecting electrode for injecting holes into said light-emitting layer; an electron injecting electrode for injecting electrons into said light-emitting layer; and a gate electrode provided opposite to said hole injecting electrode and said electron injecting electrode for controlling the distribution of the carriers in said light-emitting layer.

6. The light-emitting transistor device of claim 5 wherein said hole injecting electrode and said electron injecting electrode have comb tooth shaped regions, respectively, which are each made of a plurality of comb teeth, and wherein the comb teeth which constitute the comb tooth shaped region of the hole injecting electrode and the comb teeth which constitute the comb tooth shaped region of the electron injecting electrode are alternately arranged at predetermined intervals.

7. A pyrene-based organic compound expressed by the below formula (3), 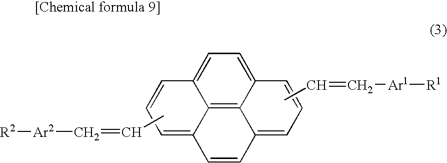
(In the formula (3), Ar¹ and Ar² represent, independently of each other, aromatic hydrocarbon groups other than a pyrene ring or aromatic heterocyclic groups. R¹ and R² represent, independently of each other, halogen atoms, alkyl groups which may have a substituent having a carbon number of 2-20 or alkoxyl groups having a carbon number of 1-20, each of Ar¹ and Ar² has one or more of R¹ or R², the abovementioned substituent is a group selected from aromatic hydrocarbon groups having a carbon number of 6-36, aromatic heterocyclic groups having a carbon number of 4-36, and halogen atoms.)

8. A pyrene-based organic compound expressed by the below formula (4), 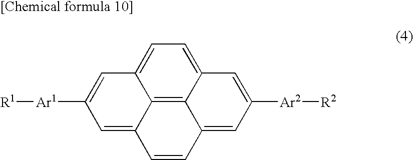
(In the formula (4), Ar¹ and Ar² represent, independently of each other, aromatic hydrocarbon groups other than a pyrene ring or aromatic heterocyclic groups, R¹ and R² represent, independently of each other, halogen atoms, alkyl groups which may have a substituent having a carbon number of 2-20 or alkoxyl groups having a carbon number of 1-20, each of Ar¹ and Ar² has one or more of R¹ or R², the abovementioned substituent is a group selected from aromatic hydrocarbon groups having a carbon number of 6-36, aromatic heterocyclic groups having a carbon number of 4-36, and halogen atoms.)

9. A pyrene-based organic compound expressed by the below formula (5), 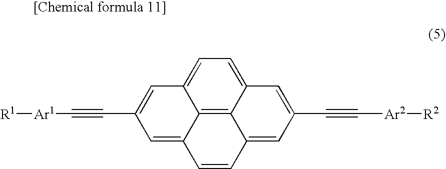
(In the formula (5), Ar¹ and Ar² represent, independently of each other, aromatic hydrocarbon groups other than a pyrene ring or aromatic heterocyclic groups, R¹ and R² represent, independently of each other, halogen atoms, alkyl groups which may have a substituent having a carbon number of 2-20 or alkoxyl groups having a carbon number of 1-20, each of Ar¹ and Ar² has one or more of R¹ or R², the abovementioned substituent is a group selected from aromatic hydrocarbon groups having a carbon number of 6-36, aromatic heterocyclic groups having a carbon number of 4-36, and halogen atoms.)
